SEQUENCE LISTING (11 sequences)

SEQ ID NO: 1 - DNA, 26 bp, Artificial sequence
  synthetic sequence
atgtcaccacaaacagagactaaagc

SEQ ID NO: 2 - DNA, 21 bp, Artificial sequence
  synthetic sequence
gaaacggtctctccaacgcat

SEQ ID NO: 3 - DNA, 20 bp, Artificial sequence
  synthetic sequence
ctcgctacaatagcagcgtc

SEQ ID NO: 4 - DNA, 22 bp, Artificial sequence
  synthetic sequence
ggaagtaaaagtcgtaacaagg

SEQ ID NO: 5 - DNA, 18 bp, Artificial sequence
  synthetic sequence
cggaccgactgcgagtcc

SEQ ID NO: 6 - DNA, 20 bp, Artificial sequence
  synthetic sequence
ctccaaaagtggtggcattg

SEQ ID NO: 7 - DNA, 19 bp, Artificial sequence
  synthetic sequence
gcaacagcaaagccgattc

SEQ ID NO: 8 - DNA, 40 bp, Artificial sequence
  synthetic sequence
tctccattaacgccgcagcagtggacgctgctattgtagc

SEQ ID NO: 9 - DNA, 41 bp, Artificial sequence
  synthetic sequence
cgtggtatggttggcttcggcatggttcaccagtccatcac

SEQ ID NO: 10 - DNA, 19 bp, Artificial sequence
  synthetic sequence
acaaaccggtcgccaactc

SEQ ID NO: 11 - DNA, 25 bp, Artificial sequence
  synthetic sequence
atgcgcttattgggtgattttcctg

(19) United States
(12) Patent Application Publication
(10) Pub. No.: US 2021/0380965 A1
(43) Pub. Date: Dec. 9, 2021

(54) MICRONEEDLE-BASED RAPID ANALYTE EXTRACTION FROM PLANT AND ANIMAL TISSUES AND RELATED METHODS AND SYSTEMS

(71) Applicant: North Carolina State University, Raleigh, NC (US)

(72) Inventors: Qingshan Wei, Cary, NC (US); Jean Beagle Ristaino, Cary, NC (US); Rajesh Paul, Raleigh, NC (US); Zhen Gu, Los Angeles, CA (US)

(21) Appl. No.: 17/342,327

(22) Filed: Jun. 8, 2021

Related U.S. Application Data
(60) Provisional application No. 63/036,098, filed on Jun. 8, 2020.

(51) Int. Cl. C12N 15/10; C12Q 1/686

(52) U.S. Cl. CPC C12N 15/1006; C12Q 1/686

(57) ABSTRACT

Methods of extracting analytes of interest from biological samples including soft plant tissues (e.g., plant leaves) or animal tissues are described. The methods include contacting the biological samples with a microneedle that absorbs the analyte of interest. Related systems and methods of detecting pathogens or pests or of genotyping the plant or animal from which the biological sample is derived are described. Also described are methods of delivering a substance of interest (e.g., a pesticide) to a plant by contacting a soft plant tissue with a microneedle coated with the substance of interest.

### CROSS-REFERENCE TO RELATED APPLICATIONS

This application claims benefit of U.S. Provisional Patent Application Ser. No. 63/036,098, filed Jun. 8, 2020, the disclosure of which is incorporated by reference herein in its entirety.

### STATEMENT OF GOVERNMENT SUPPORT

This invention was made with government support under grant number 2015-68004-23179 awarded by the United States Department of Agriculture's National Institute of Food and Agriculture (USDA/NIFA). The government has certain rights in the invention.

### TECHNICAL FIELD

The subject matter described herein relates to methods and systems for extracting analytes from biological samples, including animal tissues and plant soft tissues (e.g., plant leaves), by contacting the biological sample with a microneedle. The subject matter also includes methods of detecting pathogens in a plant or animal or genotyping the plant or animal. In addition, the subject matter relates to methods of delivering a substance of interest to a plant.

### BACKGROUND

Global food security is a growing concern both locally and internationally as it directly affects the economy and sustainability.¹ The demand of food is increasing rapidly due to population growth. By 2050, the global food production needs to be increased by 70-100% to fulfill the food demand of the growing population.² Additionally, the total available agricultural land area is decreasing with population growth. As a result, in the coming decades increasing agricultural productivity will be important to global food security. Plant and animal diseases significantly affect agricultural productivity. Crop failure due to pathogen infection is a common issue in agriculture. Every year, crop losses exceeding more than 30% occur due to plant diseases.³ Current crop protection relies on several disease diagnosis technologies, such as isolation and culture of pathogens, polymerase chain reaction (PCR), and enzyme-linked immunosorbent assay (ELISA).⁴⁻⁶ However, most of existing disease detection technologies are laboratory-based, and need well-equipped laboratories and skilled technicians. Moreover, field sites are often far from centralized diagnostic laboratories or in resource-limited regions where diagnostic facilities are not readily available. As such, there is an ongoing need for the development of field-portable and cost-effective disease detection systems and related detection methods for global crop protection.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a method of extracting an analyte from a biological sample comprising a plant tissue or an animal tissue, wherein when the biological sample comprises a plant tissue, the plant tissue is a soft plant tissue; the method comprising: contacting the plant tissue or the animal tissue with a microneedle, wherein said microneedle comprises a body having a base and a tip; removing the microneedle from contact with the plant tissue or the animal tissue, wherein the removed microneedle comprises absorbed analyte extracted from the plant tissue or the animal tissue. In some embodiments, the method further comprises collecting the analyte. In some embodiments, the soft plant tissue is selected from the group comprising an immature plant tissue, a flower, a seedling, a plant leaf, a tuber, a fruit and a stem.

In some embodiments, the microneedle has a fracture force of about 1 newton (N) or less. In some embodiments, the fracture force is about 0.1 N to about 1 N. In some embodiments, the microneedle body has a length of about 50 microns (μm) to about 1500 μm; and wherein the tip has a diameter of about 1 μm to about 10 μm. In some embodiments, the microneedle has a length of about 800 μm and a tip diameter of about 5 μm. In some embodiments, the microneedle body has a tapered, conical, and/or pyramidal shape, wherein said microneedle body is hollow or solid, and wherein the base of the microneedle has a diameter that is greater than the diameter of the tip of the microneedle. In some embodiments, the base has a diameter that is about 10 μm to about 500 μm. In some embodiments, the base has a diameter that is about 150 μm.

In some embodiments, the body of the microneedle comprises, consists essentially of, or consists of a polymer. In some embodiments, the polymer is a swellable and/or hydrophilic polymer. In some embodiments, the body of the microneedle comprises, consists essentially of, or consists of a hydrophilic, swellable polymer selected from the group comprising polyvinyl alcohol (PVA), crosslinked hyaluronic acid (HA), crosslinked polyacrylic acid (PAA), chitosan, and a copolymer thereof.

In some embodiments, the polymer is a charge-switchable polymer that is positively charged when exposed to a first pH range and uncharged when exposed to a second pH range. In some embodiments, the charge-switchable polymer is positively charged when exposed to a pH below about 7. In some embodiments, said polymer is chitosan.

In some embodiments, the method is free of the use of suction. In some embodiments, the analyte comprises a DNA, a RNA, a protein, carbohydrate or other chemical constituent and/or a small molecule, wherein said DNA, RNA, and/or protein is native to the plant tissue or animal tissue and/or to a pathogen or pest.

In some embodiments, the contacting comprises: placing the tip of the microneedle on an outer surface of the plant tissue or the animal tissue; and exerting a force on the microneedle sufficient to puncture the outer surface of the plant tissue or the animal tissue with the tip of the microneedle, thereby bringing at least a portion of the body of the microneedle into contact with inner cells of the plant tissue or the animal tissue. In some embodiments, the outer surface is a cuticle layer or an epidermal layer of a plant tissue or a skin surface of an animal. In some embodiments, collecting the analyte comprises: contacting the microneedle with a liquid in which the analyte is soluble, thereby dissolving the absorbed analyte in the liquid and removing it from the microneedle; and collecting the liquid comprising the dissolved analyte. In some embodiments, the analyte comprises one or more nucleic acid and the liquid is a nucleic acid extraction buffer solution. In some embodiments, the liquid is Tris-EDTA or nuclease-free water.

In some embodiments, contacting with a microneedle comprises contacting the outer surface of the plant tissue or the animal tissue with a plurality of microneedles. In some embodiments, the plurality of microneedles is provided in an array format.

In some embodiments, the method is performed in about 1 minute or less.

In some embodiments, the method further comprises analyzing the analyte. In some embodiments, the analyzing comprises identifying, sequencing, and/or quantifying the analyte.

In some embodiments, the biological sample comprises a plant tissue, optionally a plant leaf. In some embodiments, said plant tissue is from a plant cultivated as part of an agricultural crop for use as food for humans or other animals, as fiber, as energy, or for the production of an industrial or consumer good.

In some embodiments, the presently disclosed subject matter provides a method of detecting a pathogen or pest in a plant or an animal, wherein the method comprises: providing a microneedle or microneedle patch comprising one or more microneedles, wherein the microneedle or each microneedle of the microneedle patch has a body with a base and a tip, optionally wherein each microneedle body comprises, consists essentially of, or consists of a polymer, optionally a swellable and/or hydrophilic polymer; contacting the microneedle or microneedle patch with a plant or animal tissue, wherein said plant tissue is a soft plant tissue; removing the microneedle or microneedle patch from contact with the plant or animal tissue, wherein the removed microneedle or one or more microneedles of the removed microneedle patch comprise a foreign analyte extracted from the plant or animal tissue, wherein said foreign analyte is associated with the pathogen or pest; and analyzing the foreign analyte to determine the presence of the pathogen or pest. In some embodiments, the soft plant tissue is a plant leaf. In some embodiments, the foreign analyte is a nucleic acid and/or protein from the pathogen or pest. In some embodiments, the method further comprises collecting the foreign analyte from the microneedle or microneedle patch.

In some embodiments, the method comprises detecting a pathogen selected from the group comprising a viral pathogen, a fungal pathogen, a bacterial pathogen, and an oomycete pathogen. In some embodiments, the pathogen is a fungal pathogen, a bacterial pathogen, or an oomycete pathogen; and the foreign analyte is DNA or RNA. In some embodiments, the pathogen is a viral pathogen and the foreign analyte is viral RNA.

In some embodiments, the foreign analyte is a nucleic acid and the analyzing comprises nucleic acid amplification. In some embodiments, the amplification comprises a technique selected from the group comprising polymerase chain reaction (PCR), reverse transcription PCR (RT-PCR), ligase chain reaction (LCR), transcription-based amplification, self-sustained sequence replication (3SR), loop-mediated isothermal amplification (LAMP), reverse transcription LAMP (RT-LAMP), nucleic acid sequence-base amplification (NASBA), rolling circle amplification (RCA), ligation-enabled padlock RCA, and CRISPR cassette. In some embodiments, the nucleic acid amplification is an isothermal amplification technique.

In some embodiments, the foreign analyte comprises a nucleic acid and the analyzing comprises sequencing and/or genotyping. In some embodiments, the genotyping comprises detection of one or more marker of interest selected from an allele; a locus; a microsatellite; a single nucleotide polymorphism (SNP); a single feature polymorphism (SFP); an insertion/deletion polymorphism; a restriction-fragment-length polymorphism (RFLP); an amplified fragment length polymorphism (AFLP); and a CRISPR cassette. In some embodiments, the locus is a quantitative trait locus (QTL). In some embodiments, the microsatellite is a short tandem repeat (STR) or a simple sequence repeat (SSR).

In some embodiments, the contacting comprises: placing the tip of the microneedle or a tip of one or more microneedles of the microneedle patch on an outer surface of the plant tissue or animal tissue; and exerting a force on the microneedle or microneedle patch sufficient to puncture said outer surface, thereby bringing at least a portion of the body of the microneedle or of one or more microneedles of the microneedle patch into contact with inner cells of the plant tissue or the animal tissue. In some embodiments, the outer surface is a cuticle or epidermal layer of the plant tissue or a skin surface of an animal. In some embodiments, the plant tissue is a plant leaf. In some embodiments, the animal is a human.

In some embodiments, collecting the foreign analyte comprises: contacting the microneedle or microneedle patch with a liquid in which the foreign analyte is soluble, thereby dissolving the absorbed foreign analyte in the liquid and removing it from the microneedle or microneedle patch; and collecting the liquid comprising the dissolved foreign analyte. In some embodiments, the analyzing is free of cell lysis and/or nucleic acid purification. In some embodiments, the microneedle patch comprises between 1 and 1,000 microneedles, inclusive. In some embodiments, the method comprises detecting a plant pathogen in a plant. In some embodiments, the plant pathogen is selected from Phytophthora infestans, Xanthomonas perforans, Altermaria linariae, and tomato spotted wilt virus (TSWV).

In some embodiments, the presently disclosed subject matter provides a method of genotyping a plant or animal, the method comprising: providing a microneedle or microneedle patch comprising one or more microneedles, wherein the microneedle or each microneedle of the microneedle patch has a body with a base and a tip; contacting the microneedle or microneedle patch with a plant or animal tissue, wherein the plant tissue is a soft tissue; removing the microneedle or microneedle patch from contact with the plant or animal tissue, wherein the removed microneedle or one or more microneedle of the removed microneedle patch comprise a native analyte, optionally a native nucleic acid and/or protein, extracted from the plant or animal tissue; and analyzing the native analyte, thereby genotyping the plant or animal. In some embodiments, each microneedle body comprises, consists essentially of, or consists of a polymer. In some embodiments, the polymer is a swellable and/or a hydrophilic polymer. In some embodiments, the soft tissue is a plant leaf. In some embodiments, the method further comprises collecting the native analyte from the microneedle or microneedle patch.

In some embodiments, a native nucleic acid is extracted from the plant or animal tissue, and the analyzing comprises nucleic acid amplification. In some embodiments, the amplification comprises a technique selected from the group comprising PCR, RT-PCR, LCR, transcription-based amplification, 3SR, LAMP, RT-LAMP, RCA, ligation-enabled padlock RCA, NASBA, and CRISPR cassette.

In some embodiments, the analyzing comprises sequencing and/or genotyping. In some embodiments, analyzing comprises detection of one or more marker of interest selected from an allele; a locus; a microsatellite; a SNP; a SFP; an insertion/deletion polymorphism; a RFLP; an AFLP; and a CRISPR cassette. In some embodiments, the locus is a QTL. In some embodiments, the microsatellite is a STR or a SSR.

In some embodiments, the contacting comprises: placing a tip of the microneedle or of one or more microneedle of the microneedle patch on an outer surface of the plant or animal tissue; and exerting a force on the microneedle or microneedle patch sufficient to puncture said outer surface, thereby bringing at least a portion of the body of the microneedle or of one or more microneedle of the microneedle patch into contact with inner cells of the plant or animal tissue. In some embodiments, the outer surface is a cuticle or epidermal layer of a plant tissue or a skin surface of an animal. In some embodiments, the plant tissue is a leaf. In some embodiments, the animal is a human. In some embodiments, collecting the native analyte comprises: contacting the microneedle or microneedle patch with a liquid in which the native analyte is soluble, thereby dissolving the native analyte in the liquid and removing it from the microneedle or microneedle patch; and collecting the liquid comprising the dissolved native analyte. In some embodiments, the analyzing is free of cell lysis and/or nucleic acid purification. In some embodiments, the microneedle patch comprises between 1 and 1,000 microneedles, inclusive.

In some embodiments, the presently disclosed subject matter provides a method of delivering a substance of interest to a plant; the method comprising: providing a microneedle or microneedle patch comprising one or more microneedles, wherein the microneedle or each microneedle of the microneedle patch comprises a body comprising a base and a tip, and wherein the body of the microneedle or the body of at least one body of the microneedle patch comprises or is coated with a substance of interest; and contacting the microneedle or microneedle patch with a plant tissue, wherein said plant tissue is a soft tissue, thereby delivering the substance of interest to the plant. In some embodiments, the microneedle patch comprises a plurality of microneedles. In some embodiments, the plant soft tissue is a plant leaf.

In some embodiments, the substance of interest comprises one or more of the group selected from a pesticide, DNA, RNA, a protein, a peptide, an antigen for stimulating the plant immune system, a vector, a plasmid, a CRISPR cassette, a biological cell, a biological cell component, and/or a vesicle. In some embodiments, the substance of interest comprises a pesticide. In some embodiments, the pesticide is selected from a systemic fungicide, a translaminar fungicide, an herbicide, an insecticide, a biological control agent, and combinations thereof.

In some embodiments, the body of each microneedle comprises, consists essentially of, or consists of a polymer. In some embodiments, the porosity of the polymer is tailored to provide a desired rate of delivery of the substance of interest or wherein said polymer is biodegradable.

In some embodiments, the presently disclosed subject matter provides a system for detecting an analyte of interest in a biological sample, the system comprising: a microneedle or microneedle patch configured to obtain an analyte extract from the sample, wherein said microneedle patch comprises one or more microneedles, wherein the microneedle or each microneedle of the microneedle patch comprises a body comprising a base and a tip; a receiver configured to receive a reaction mixture comprising (i) one or more reagents for detection of an analyte from the sample and (ii) the microneedle, the microneedle patch or an extract from the biological sample obtained using the microneedle or microneedle patch; and a detector for detecting a signal from the reaction mixture associated with a reaction or interaction between an analyte and at least one of the one or more reagents; wherein the biological sample is an animal and the microneedle or microneedle patch is configured to obtain an analyte extract from the animal by insertion and removal from a skin surface of the animal; or wherein the biological sample is a soft tissue from a plant and the microneedle or microneedle patch is configured to obtain an analyte extract from the plant by insertion and removal from the soft tissue of the plant, optionally wherein the soft tissue is a plant leaf. In some embodiments, the analyte of interest comprises a native analyte or a foreign analyte associated with a pathogen or pest, or any combination thereof. In some embodiments, the microneedle patch comprises a plurality of microneedles configured in an array. In some embodiments, each microneedle body comprises, consists essentially of, or consists of a swellable and/or hydrophilic polymer.

In some embodiments, the analyte extract comprises a nucleic acid, and wherein the one or more reagents comprise a polymerase and one or more nucleic acid primers for amplification of a nucleic acid associated with the one or more nucleic acids of interest. In some embodiments, the analyte extract comprises a RNA, and wherein the one or more reagents further comprise a reverse transcriptase. In some embodiments, the one or more reagents further comprise one or more fluorescent or colorimetric dyes. In some embodiments, the one or more fluorescent or colorimetric dyes comprise hydroxynaphthol blue (HNB).

In some embodiments, the system further comprises an attachment configured to position the receiver with respect to the detector. In some embodiments, the detector comprises a camera configured to capture an image of the receiver or a portion thereof. In some embodiments, the detector comprises a consumer electronics device having a camera configured to capture an image of the receiver and one or more light sources configured to illuminate the receiver. In some embodiments, the consumer electronics device is a smartphone or a tablet. In some embodiments, the one or more light sources comprise one or more light emitting diodes (LEDs) or a laser diode. In some embodiments, the receiver comprises a polydimethylsiloxane (PDMS) chamber or chip.

In some embodiments, the system further comprises a heating device configured to heat the receiver to a pre-determined temperature for a pre-determined period of time. In some embodiments, the heating device is a flexible, polyamide heating device or a self-heating device or pad. In some embodiments, the system further comprises one or more battery. In some embodiments, the one or more battery is configured to provide power to a heating device configured to heat the receiver and/or one or more light source associated with the detector.

In some embodiments, the presently disclosed subject matter provides a method of detecting a presence and/or an amount of one or more analyte of interest in a biological sample, wherein the method comprises use of the presently disclosed system. In some embodiments, the one or more analytes of interest are associated with a pathogen or pest.

Accordingly, it is an object of the presently disclosed subject matter to provide a method of extracting an analyte from a plant soft tissue or animal tissue, detecting a pathogen of pest in such a tissue, genotyping such a tissue, delivering a substance to a plant soft tissue, and to a related system.

An object of the presently disclosed subject matter having been stated hereinabove, and which is achieved in whole or in part by the compositions and methods disclosed herein, other objects will become evident as the description proceeds when taken in connection with the accompanying Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Reference will now be made to the accompanying drawings, which are not necessarily drawn to scale.

FIGS. 1A-1E: Microneedle (MN)-based rapid extraction of plant DNA. FIG. 1A is a schematic drawing of different steps for conventional cetyltrimethylammonium bromide (CTAB) extraction (top) and MN extraction (bottom). FIG. 1B is a scanning electron microscope (SEM) image of a MN patch of the presently disclosed subject matter. The scale bar in the bottom right represents 300 micrometers (m). FIG. 1C is a photograph of a MN patch of the presently disclosed subject matter. The scale bar in the bottom right represents 5 millimeters (mm). FIGS. 1D and 1E are ultraviolet (UV) absorption spectra of DNA solutions extracted by the MN patch (FIG. 1D), CTAB protocol (FIG. 1E, dashed lines), sodium dodecyl sulfate (SDS) protocol (FIG. 1E, dotted lines), and sodium hydroxide (NaOH) protocol (FIG. 1E, solid lines), respectively. The dotted line in FIG. 1D represents data from a rinsing solution from a blank MN patch without leaf puncturing.

FIGS. 2A and 2B: Direct polymerase chain reaction (PCR) amplification of microneedle (MN)-extracted DNA without purification. FIG. 2A is a graph of DNA extraction (in nanograms per microliter (ng/μL) by MN patches from tomato (left), potato (middle), and pepper leaves (right) (N=5), indicating the applicability of the MN extraction method for different plant species. FIG. 2B is a pair of gel electrophoresis images showing amplified bands of ribulose-bisphosphate carboxylase (rbcL) gene using MN-extracted DNA from (left) tomato and (right) pepper leaves, respectively. Lanes 1-5 in each of the gels represent 5 replicates using different samples; L: 100 basepair (bp) DNA ladder; NC: negative control (no DNA: blank rinsing solutions).

FIGS. 3A and 3B: Detection of P. infestans via microneedle (MN) extraction from laboratory-inoculated samples. FIG. 3A is a schematic drawing and a gel electrophoresis image showing the amplified bands of P. infestans from cetyltrimethylammonium bromide (CTAB)-extracted DNA. FIG. 3B is a pair of schematic drawings and gel electrophoresis images of amplified bands of P. infestans from MN-extracted DNA. At left is a schematic drawing and gel electrophoresis image of MN-extracted DNA where the surface of the leaves was disinfested using 5% bleach solution for a few seconds before extraction. At right is a schematic drawing and gel electrophoresis image showing the amplified bands of P. infestans from extraction solutions obtained by flat PVA patches (no microneedles). Lanes 1-6 in gel electrophoresis images of FIGS. 3A and 3B, left: 6 replicates extracted by the CTAB and MN patch methods, respectively; Lanes 1-2 in FIG. 3B, right: flat PVA patch extraction after surface disinfestation using a 5% bleach solution; Lanes 3-4 in FIG. 3B, right: flat PVA patch extraction without bleach disinfesting; L:100 bp ladder; NC: negative control (no DNA: blank rinsing solutions); PC: positive control (purified P. infestans DNA). All leaves were tested 4 days after inoculation.

FIGS. 4A-4D: Application of microneedle (MN) extraction for detection of P. infestans from field-collected samples. FIG. 4A is a series of photographic images of infected tomato leaves; the squares indicate DNA extraction areas for late blight disease detection. FIGS. 4B and 4C are graphs of real-time polymerase chain reaction (PCR) amplification curves (relative fluorescence units (RFU) in arbitrary units (a.u.) versus cycle number) using (FIG. 4B) MN-extracted and (FIG. 4C) cetyltrimethylammonium bromide CTAB-extracted DNA, respectively. FIG. 4D is a graph showing a comparison of cycle threshold (Ct) values between the two methods (MN patch, left; and CTAB, right) for detection of P. infestans in field samples.

FIGS. 5A-5D: Loop-mediated isothermal amplification (LAMP) assay for the detection of P. infestans. FIG. 5A is a graph of real-time amplification curves (relative fluorescence units versus time in minutes) of P. infestans DNA at different concentrations (1.1 picograms per microliter (pg/μL), 11 pg/μL, 110 pg/μL, 1.1 nanograms per microliter (ng/μl), or 11 ng). FIG. 5B is a graph of melt curve analysis of the amplicons as described from FIG. 5A, showing identical products. FIG. 5C is a graph of a standard curve (threshold cycle versus log of the concentration of DNA (in nanograms per microliter (ng/μL)) for the real-time P. infestans LAMP assay. FIG. 5D is a series of photographic images of colorimetric detection of LAMP amplification from the color change of the hydroxynaphthol blue (HNB) dye. Images correspond to results from 11 nanograms per microliter (ng/μL) DNA (top left); 1.1 ng/μL (top right); 101 picograms per microliters (pg/μL) (middle left); 11 pg/μL (middle right); 1.1 pg/μL (bottom left); and a negative control sample (NTC).

FIGS. 6A-6C: Direct loop-mediated isothermal amplification (LAMP) assay of microneedle (MN)-extracted DNA without purification. FIG. 6A is a graph of the real-time LAMP amplification curves (relative fluorescence units (RFU) in arbitrary units (a.u.) versus time in minutes (min)) for MN extraction and cetyltrimethylammonium bromide (CTAB) extraction. FIG. 6B is a pair of photographs showing colorimetric detection of LAMP amplicons based on the color change of hydroxynaphthol blue (HNB) dye. The photograph at the top includes NM extracted and CTAB extracted samples, and the photograph at the bottom includes positive and negative controls. FIG. 6C is a gel electrophoresis image showing amplified LAMP products. Lane 3-6 represent 4 replicates of MN extraction using different leaves; Lane 8-11 represent 4 different replicates of CTAB extraction using different leaves; Lane 2 and 7 are 100 basepair (bp) DNA ladder; Lane 1 and 13 are negative control (no DNA and MN-extracted DNA from healthy leaf); Lane 12 is positive control.

FIGS. 7A-7C: Loop-mediated isothermal amplification (LAMP) assay by dipping microneedle (MN) patch into the LAMP master mix. FIG. 7A is a schematic drawing of a LAMP assay process that includes dipping a MN patch in a LAMP master mix after punctuation of a leaf sample. After peeling the MN patch off the leaf sample, a small piece of MN patch was dipped into the LAMP master mix to deposit template DNA for running LAMP amplification. This modification eliminates the rinsing step of the MN patch with tris(hydroxymethyl)aminomethane-ethylenediaminetetraacetic (TE) buffer. FIG. 7B is a pair of photographs showing visualization of successful LAMP amplification from the color change of hydroxynaphthol blue (HNB) dye. S1-S4 are replicates of modified MN extraction using different pathogen infected leaves; PC1 and PC2 are positive controls (CTAB-extracted DNA from an infected leaf); and NC1 and NC2 are negative controls (MN-extracted DNA from a healthy leaf). FIG. 7C is an image of gel electrophoresis showing amplified LAMP products. Lane 2-5 represent 4 replicates of modified MN extraction using different leaves; Lane 1 is 100 basepair (bp) DNA ladder; Lane 6 is positive control (1 μL CTAB extracted DNA from infected leaf); Lane 7 is negative control (MN-extracted DNA from healthy leaf).

FIGS. 8A-8D: P. infestans detection by loop-mediated amplification (LAMP) assay in presence of polyvinyl alcohol (PVA) microneedle (MN) patches. FIGS. 8A and 8B are photographs of LAMP reaction solutions in the presence of PVA MN patches (FIG. 8A) before amplification and (FIG. 8B) after amplification. FIG. 8C is a photograph of negative control solutions of the LAMP reaction showing no color change in the absence of a MN patch. FIG. 8D is a gel electrophoresis image showing amplified LAMP products. Lane 1-8 represent eight different replicates of LAMP amplification in presence of PVA MN patches; Lane 9 represent the negative control in the absence of MN patch.

FIGS. 9A-9D: Smartphone-based loop-mediated amplification (LAMP) platform. FIG. 9A is a schematic drawing of a smartphone reader device and a LAMP sample cassette. FIG. 9B is a schematic drawing of a cross-section view of the LAMP reader device shown in FIG. 9A, showing its internal components. FIG. 9C is a photographic image of a flexible polyamide heater used to heat the LAMP reaction in a LAMP smartphone reader device. FIG. 9D is a graph of heater surface temperatures (in degrees Celsius (° C.) versus time (in minutes (min)) at two different input voltages (5.25 volts (V) (0.26 ampere (A)) and 6.25 V (0.32 A)).

FIGS. 10A-10C: FIGS. 10A and 10B show a comparison of real-time loop-mediated amplification (LAMP) assay obtained by (FIG. 10A) a benchtop thermal cycler and (FIG. 10B) a smartphone-based LAMP reader device of the presently disclosed subject matter. Normalized fluorescence results (as a function of time in minutes (min)) are shown for samples of P. infestans DNA at 1 nanograms per microliter (ng/μL); 100 picograms per microliter (pg/μL), 10 pg/μL, and 1 nanograms per microliter (ng/μL). FIG. 10C is a graph of LAMP standard curves (threshold time (Tt) in minutes versus log DNA concentration (ng)) obtained from 2 different platforms (benchtop real-time detection device, squares; smartphone-based reader device, circles) for the detection of P. infestans.

FIGS. 11A-11C: Extraction of total RNA for the detection of tomato spotted wilt virus (TSWV) in Emilia and tomato plant leaves. FIG. 11A is a pair of photographs of TSWV-infected Emilia (top) and tomato (bottom) plant leaves. FIG. 11B is a graph (relative fluorescence units versus cycle number) of reverse transcription-polymerase chain reaction (RT-PCR) amplification of TSWV RNA extracted by a spin column-based RNA isolation kit (sold under the tradename RNEASY™ mini kit, Qiagen GMBH, Hilden, Germany), a conventional extraction protocol using a reagent sold under the tradename TRIZOL™ (Molecular Research Center, Inc., Cincinnati, Ohio, United States of America), and a microneedle (MN) patch of the presently disclosed subject matter. FIG. 11C is a graph (relative fluorescence units versus time in minutes (min)) of reverse transcription loop-mediated isothermal amplification (RT-LAMP) of TSWV RNA extracted by a spin column-based RNA isolation kit (sold under the tradename RNEASY™ mini kit, Qiagen GMBH, Hilden, Germany), a conventional extraction protocol using a reagent sold under the tradename TRIZOL™ (Molecular Research Center, Inc., Cincinnati, Ohio, United States of America), and a microneedle (MN) patch of the presently disclosed subject matter.

FIG. 12 is a graph of the average sporangia count (per milliliter (mL)) over three repetitions of fungicide (sold under the tradename PRESIDIO™ (Valent U.S.A., Walnut Creek, Calif., United States of America)) or buffer treatments applied to detached tomato leaves before the introduction of P. infestans sporangia. Sporangia counts were measured seven days post-inoculation. Fungicide/Buffer was applied by a microneedle patch of the presently disclosed subject matter or applied via spray.

FIGS. 13A-13D: Photographs of examples of leaves treated with a fungicide (sold under the tradename PRESIDIO™ (Valent U.S.A., Walnut Creek, Calif., United States of America)) or buffer seven days after inoculation with P. infestans sporangia. FIG. 13A is a photograph of a leaf treated with sprayed buffer. FIG. 13B is a photograph of a leaf treated with buffer applied via a microneedle (MN) patch of the presently disclosed subject matter. FIG. 13C is a photograph of a leaf treated with sprayed fungicide. FIG. 13D is a photograph of a leaf treated with fungicide applied via a MN patch of the presently disclosed subject matter.

FIG. 14 is a graph of the average sporangia counts (per milliliter (mL)) over three repetitions for two experiments of a fungicide (sold under the tradename RIDOMIL GOLD™ (Syngenta Participations AG Corporation, Basel, Switzerland)) or buffer treatments applied to detached tomato leaves before the introduction of P. infestans sporangia. Sporangia counts were measured seven days post-inoculation. Fungicide/Buffer was applied by a microneedle patch of the presently disclosed subject matter or applied via spray.

FIGS. 15A-15D: Photographs of examples of leaves treated with ether a fungicide (sold under the tradename RIDOMIL GOLD™ (Syngenta Participations AG Corporation, Basel, Switzerland)) or buffer seven days after inoculation with P. infestans sporangia. FIG. 15A is a photograph of a leaf treated with sprayed buffer. FIG. 15B is a photograph of a leaf treated with buffer applied via microneedle (MN) patch. FIG. 15C is a photograph of a leaf treated with sprayed fungicide. FIG. 15D is a photograph of a leaf treated with fungicide applied via MN patch.

FIG. 16 is a graph of average sporangia counts (per milliliter (mL)) of eight treatments of a fungicide (sold under the tradename RIDOMIL GOLD™ (Syngenta Participations AG Corporation, Basel, Switzerland)) or buffer using application via either a microneedle (MN) patch (black bars) or spray (grey bars). Sporangia counts were measured seven days post inoculation and are averaged over three plates.

FIGS. 17A-17D: Photographs of examples of leaves treated with buffer or with increasing dosages of a fungicide (sold under the tradename RIDOMIL GOLD™ (Syngenta Participations AG Corporation, Basel, Switzerland)) Leaves were treated using either a spray solution or with a microneedle (MN) patch application. Dosages of fungicide were at 0 (i.e., buffer, FIG. 17A), a quarter of the standard application rate (FIG. 17B), half-standard application rate (FIG. 17C), and full standard application rate (FIG. 17D).

FIGS. 18A and 18B: Simultaneous detection of multiple plant pathogens in a single leaf using an integrated microneedle (MN)-smartphone loop-mediated amplification (LAMP) platform. FIG. 18A is (top) a series of representative smartphone fluorescence images of the multiplexed LAMP assay chip for healthy tomato leaves (left), P. infestans (second from left), tomato spotted wilt virus (TSWV, second from right), or co-infection (P. Infestans plus TSWV, right); and (bottom) a series of graphs of the normalized fluorescence intensities of different reaction chambers in the LAMP assay chip shown above the graph. In the images at the top, location I=rbcl gene; Location II=P. infestans; Location III=TSWV FIG. 18B is a series of photographs of representative leaf samples from each test group used in the analyses described for FIG. 18A. All Error bars indicate SD for N=6 samples (two separate trials with 3 leaves for each test group in each trial).

### REFERENCE TO SEQUENCE LISTING

The Sequence Listing associated with the instant disclosure has been submitted electronically herewith as an 3 kilobyte file with File Name (297-335-2.ST25.txt), Creation Date (Jun. 8, 2021), Computer System (IBM-PC/MS-DOS/MS-Windows), and Docket No. (297/335/2). The Sequence Listing submitted electronically herewith is hereby incorporated by reference into the instant disclosure.

### DETAILED DESCRIPTION

The presently disclosed subject matter relates, in some aspects, to a microneedle (MN)-based nucleic acid (or other analyte of interest) extraction method, a MN-based substance delivery method for plants, and to the development of a multifunctional molecular diagnostic platform for nucleic acid (or other analyte) extraction, amplification, detection, and/or delivery of substances (e.g., fungicides or other pesticides) to plants, all with the same device. The presently disclosed MN-based nucleic acid extraction approach does not rely on the conventional concept of nucleic acid isolation through tissue and cell lysis. Rather, it utilizes the micro-punctuation of a MN patch onto leaf tissue (or other soft plant or animal tissue) to extract intracellular DNA or RNA without the need of cell lysis. By using MN-patch extraction, the timeline of plant DNA/RNA extraction can be shortened from ˜3-4 hours in a conventional plant nucleic acid extraction method to about 1 minute or less and can overcome the burden of using bulky and expensive equipment for plant sample preparation. The presently disclosed subject matter demonstrates that the MN-extracted DNA and RNA is directly applicable for subsequent molecular analysis, such as polymerase chain reaction (PCR), loop-mediated amplification (LAMP), genotyping or sequencing. Furthermore, the presently disclosed subject matter relates, in some embodiments, to combining the rapid MN extraction with a handheld smartphone device for performing amplification assays (e.g., isothermal amplification assays) without benchtop equipment. Finally, as further disclosed herein, the presently disclosed MN patches demonstrate efficient delivery of fungicides or other therapeutic agents to plant leaves for disease control.

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

As used herein and in the appended claims, the singular forms “a,” “an,” and “the” include plural referents unless the context clearly dictates otherwise. For example, an analyte refers to one or more analytes. As such, the terms “a”, “an”, “one or more” and “at least one” can be used interchangeably. Similarly, the terms “comprising”, “including” and “having” can be used interchangeably. It is further noted that the claims can be drafted to exclude any optional element. As such, this statement is intended to serve as antecedent basis for use of such exclusive terminology as “solely,” “only” and the like, in connection with the recitation of claim elements, or use of a “negative” limitation.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of concentration, volume, weight, length, width, diameter, thickness, temperature, enzymatic activity, pH, time, mass ratio, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4).

The terms “optional” and “optionally” as used herein indicate that the subsequently described event, circumstance, element, and/or method step may or may not occur and/or be present, and that the description includes instances where said event, circumstance, element, or method step occurs and/or is present as well as instances where it does not.

The term “micro” (e.g., in “microneedle”) as used herein refers to a structure having at least one region with a dimension of less than about 1,000 microns (μm). In some embodiments, the term “micro” refers to a structure having a dimension (e.g., a length, width or diameter) between about 1 micron and about 1,000 microns. In some embodiments, the term “micro” refers to a structure having a dimension between about 10 microns and about 500 microns (e.g., about 10, 25, 50, 75, 100, 150, 200, 250, 300, 350, 400, 450 or about 500 microns).

The term “diameter” is used herein to refer to the linear distance between two points on the outer surface of a named portion of a microneedle (e.g., a microneedle tip). When the cross-section of a microneedle tip is circular, the diameter can be the diameter of the circle. However, in some embodiments, the cross-section of the tip, base, or body of a microneedle can be a shape other than a circle (e.g., an oval, a square, a rectangle, etc.) and the diameter can refer to the distance between two points on the surface of the named part. In some embodiments, the diameter refers to the largest linear distance between two points on the outer surface of the named portion of the microneedle.

The terms “polymer” and “polymeric” refer to chemical structures that have repeating units (i.e., multiple copies of a given chemical substructure or “monomeric unit”). As used herein, polymers can, in some embodiments, refer to structures having more than 3, 4, 5, 6, 7, 8, 9, or 10 repeating units and/or to structures wherein the repeating unit is other than methylene. Polymers can be formed from polymerizable monomers. A polymerizable monomer is a molecule that comprises one or more reactive moieties {e.g., siloxy ethers, hydroxyls, amines, vinylic groups (i.e., carbon-carbon double bonds), halides (i.e., Cl, Br, F, and I), esters, carboxylic acids, activated esters, and the like} that can react to form bonds with other molecules. Generally, each polymerizable monomer molecule can bond to two or more other molecules. In some cases, a polymerizable monomer will bond to only one other molecule, forming a terminus of the polymeric material. Some polymers contain biodegradable linkages, such as esters or amides, such that they can degrade overtime under biological conditions.

A “copolymer” refers to a polymer derived from more than one species of monomer.

As used herein, a “block copolymer” refers to a copolymer that comprises blocks (i.e., polymeric sub-sections of the whole copolymer) in a linear sequence. A “block” refers to a portion of a copolymer that has at least one feature that is not present in the adjacent portions of the macromolecule. Thus, a “block copolymer” can refer to a copolymer in which adjacent blocks are constitutionally different, i.e., each of these blocks comprises constitutional units derived from different characteristic species of monomer or with different composition or sequence distribution of constitutional units.

“Biocompatible” as used herein, generally refers to a material and any metabolites or degradation products thereof that are generally non-toxic to the recipient and do not cause any significant adverse effects to the recipient.

“Biodegradable” means materials that are broken down or decomposed by natural biological processes. Biodegradable materials, when introduced to a biologic fluid, are broken down by cellular machinery, proteins, enzymes, hydrolyzing chemicals, reducing agents, intracellular constituents, and the like into components that the cells can either reuse or dispose of without significant toxic effect on the cells (i.e., fewer than about 20% of the cells are killed). The term “biodegradable” as used herein refers to both enzymatic and non-enzymatic breakdown or degradation of the polymeric structure. Biodegradation can take place intracellularly or intercellularly. In certain preferred embodiments, the chemical reactions relied upon to break down the biodegradable compounds are uncatalyzed. In some embodiments, the degradation time is a function of polymer composition and morphology. Suitable degradation times are from hours or days to weeks.

The term “hydrophilic” can refer to a group that can form attractive interactions (e.g., hydrogen bonding interactions) with water and/or aqueous solutions and thus be “wetted” by water and/or aqueous solutions.

The term “hydrophobic” refers to groups that do not significantly form attractive interactions and/or are repelled by water and/or aqueous solutions. In some embodiments, the term “hydrophobic” refers to groups that which preferentially form attractive interactions with fats and/or non-aqueous solutions.

The term “amphiphilic” refers to a molecule or polymer that contains both hydrophilic and hydrophobic groups.

The term “analyte” as used herein refers to a material that is extracted from a biological tissue (e.g., a soft plant tissue or an animal tissue) using a microneedle or a microneedle array. Typically, such analytes can be used to determine information about the tissue or its parent organism, such as the genotype or health of the parent organism (e.g., the presence of disease or infection). Analytes can include any chemical constituents present in the tissue, or fragments thereof, including cellular constituents that are native to the tissue (i.e., constituents that are naturally present in the tissue and/or are produced by cells in the tissue) and foreign constituents (e.g., constituents that are present in the tissue as a result of infection by a pathogenic organism, infestation by a pest, or as the result of human intervention or activity). Thus, the term analyte includes “native analytes”, i.e., cellular constituents native or endogenous to the plant or animal tissue being contacted with a microneedle, including but not limited to, nucleic acids (i.e., RNA or DNA), proteins, peptides, carbohydrates, sugars, lipids, cell wall components, membrane components, signaling molecules, and the like; and “foreign analytes”, including cellular constituents from biological organisms other than the organism that produced the tissue (e.g., pathogenic microorganisms, pests (e.g., insect pests) or biological organisms used to control pathogenic microorganisms or pests), which can include, but are not limited to, nucleic acids, proteins, peptides, carbohydrates, sugars, lipids, cell membrane components, and/or toxins (e.g., mycotoxins), as well as synthetic materials, such as a drug, a chemical pesticide or the metabolites thereof.

“Protein” refers to a polymer of amino acids. Typically, “protein” refers to longer amino acid sequences, e.g., containing at least 25, 50, 100, 200, 500 or more monomeric units, while “peptide” refers to shorter amino acid sequences.

“Polynucleotide,” “polynucleotide sequence,” “nucleic acid sequence,” “nucleic acid” and “oligonucleotide” are used interchangeably herein to indicate a polymer of nucleotides that is single- or multi-stranded, that optionally contains synthetic, non-natural, or altered RNA or DNA nucleotide bases. Thus, the term “nucleic acid” as used herein can refer to either RNA or DNA. A DNA polynucleotide can be comprised of one or more strands of cDNA, genomic DNA, synthetic DNA, or mixtures thereof.

Thus, as used herein, the term “nucleic acid” refers to deoxyribonucleotides or ribonucleotides and polymers thereof in either single or double stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed base and/or deoxyinosine residues (Batzer et al. (1991) Nuc Acids Res 19:5081; Ohtsuka et al. (1985) J Biol Chem 260:2605-2608; Rossolini et al. (1994) Mol Cell Probes 8:91-98). The terms “nucleic acid” or “nucleic acid sequence” can also be used interchangeably with gene, cDNA, and mRNA encoded by a gene.

“RNA” means ribonucleic acid that synthesizes protein within a cell, transferring information from DNA to the protein-forming system of the cell. RNA is also involved in expression and repression of hereditary information.

As used herein, “genetic element” or “gene” refers to a heritable sequence of DNA, e.g., a genomic sequence, with functional significance. The term “gene” can also be used to refer to, e.g., a cDNA and/or an mRNA encoded by a genomic sequence, as well as to that genomic sequence.

As used herein, the phrase “oligonucleotide” refers to a polymer of nucleotides of any length. In some embodiments, an oligonucleotide is a primer that is used in a polymerase chain reaction (PCR) and/or reverse transcription-polymerase chain reaction (RT-PCR), and the length of the oligonucleotide is typically between about 15 and 30 nucleotides. In some embodiments, the oligonucleotide is present on an array and is specific for an analyte (e.g., a gene) of interest. In whatever embodiment that an oligonucleotide is employed, one of ordinary skill in the art is capable of designing the oligonucleotide to be of sufficient length and sequence to be specific for the gene of interest (i.e., that would be expected to specifically bind only to a product of the gene of interest under a given hybridization condition).

As used herein, “genotype” is the genetic constitution of an individual (or group of individuals) at one or more genetic loci, as contrasted with the observable trait (phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome. Thus, in some embodiments, a genotype can represent a single locus and in others it can represent a genome-wide set of loci. In some embodiments, the genotype can reflect the sequence of a portion of a chromosome, an entire chromosome, a portion of the genome, and the entire genome. The term “genotype” can also refer to determining the genetic constitution of an individual (or group of individuals) at one or more genetic loci. A genotype can be indirectly characterized using markers or directly characterized by nucleic acid sequencing. Suitable markers include a phenotypic character, a metabolic profile, a genetic marker, or some other type of marker.

As used herein, the terms “phenotype,” or “phenotypic trait,” or “trait” refers to one or more detectable characteristics of a cell or organism which can be influenced by genotype. The phenotype can be observable to the naked eye, or by any other means of evaluation known in the art, e.g., microscopy, biochemical analysis, genomic analysis, an assay for a particular disease tolerance, etc. In some cases, a phenotype is directly controlled by a single gene or genetic locus, e.g., a “single gene trait.” In other cases, a phenotype is the result of several genes.

As used herein, “locus” refers to a chromosome region or chromosomal region where a polymorphic nucleic acid, trait determinant, gene, or marker is located. A locus can represent a single nucleotide, a few nucleotides or a large number of nucleotides in a genomic region. The loci comprise one or more polymorphisms in a population (e.g., alternative alleles are present in some individuals).

As used herein, “allele” refers to an alternative nucleic acid sequence at a particular locus. The length of an allele can be as small as one nucleotide base. For example, a first allele can occur on one chromosome, while a second allele occurs on a second homologous chromosome, e.g., as occurs for different chromosomes of a heterozygous individual, or between different homozygous or heterozygous individuals in a population.

As used herein, “polymorphism” means the presence of one or more variations in a population. A polymorphism can manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation can comprise, but is not limited to, one or more nucleotide base changes, the insertion of one or more nucleotides or the deletion of one or more nucleotides. A polymorphism can arise from random processes in nucleic acid replication, through mutagenesis, as a result of mobile genomic elements, from copy number variation and during the process of meiosis, such as unequal crossing over, genome duplication, and chromosome breaks and fusions. The variation can be commonly found or can exist at low frequency within a population, the former having greater utility in general plant breeding and the latter can be associated with rare but important phenotypic variation. Useful polymorphisms include, but are not limited to, a single nucleotide polymorphisms (SNP), an insertion or deletion in DNA sequence (indel), a simple sequence repeats of DNA sequence (SSR), a restriction fragment length polymorphism (RFLP), and a tag SNP. A genetic marker, a gene, a DNA-derived sequence, a RNA-derived sequence, a promoter, a 5′ untranslated region of a gene, a 3′ untranslated region of a gene, microRNA, small interfering RNA, a tolerance locus, a satellite marker, a transgene, mRNA, double-stranded RNA, a transcriptional profile, and a methylation pattern can also comprise a polymorphism. In addition, the presence, absence, or variation in copy number of the preceding can comprise a polymorphism.

As used herein, the term “marker” can refer to a polymorphic nucleic acid sequence or nucleic acid feature. In a broader aspect, a “marker” can be a detectable characteristic that can be used to discriminate between heritable differences between organisms. Examples of such characteristics include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics.

Methods for detecting a polymorphism at a particular locus include the use of “marker assays”, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc.

In some embodiments, “genotyping” refers to any method whereby the specific allelic form of a genomic polymorphism is determined. For example, a single nucleotide polymorphism (SNP) is typed by determining which nucleotide is present (i.e. an A, G, T, or C). Insertion/deletions (Indels) are determined by determining if the Indel is present. Indels can be typed by a variety of assays including, but not limited to, marker assays.

As used herein, the term “single nucleotide polymorphism,” also referred to by the abbreviation “SNP,” means a polymorphism at a single site wherein the polymorphism constitutes a single base pair change.

As used herein, “resistance” and “enhanced resistance” are used interchangeably herein and refer to any type of increase in resistance, or any type of decrease in susceptibility. A plant or plant variety exhibiting resistance need not possess absolute or complete resistance. Instead, a plant or plant variety with “enhanced resistance” will have a level of resistance which is higher than that of a comparable susceptible plant or variety.

As used herein, “quantitative trait locus” (QTL) or “quantitative trait loci” (QTLs) refer to a genetic domain that effects a phenotype that can be described in quantitative terms and can be assigned a “phenotypic value” which corresponds to a quantitative value for the phenotypic trait.

As used herein, “transgenic” means a plant or seed whose genome has been altered by the stable integration of recombinant DNA. A transgenic line includes a plant regenerated from an originally-transformed plant cell and progeny transgenic plants from later generations or crosses of a transformed plant.

The term “amplifying” in the context of nucleic acid amplification is any process whereby additional copies of a selected nucleic acid (or a transcribed form thereof) are produced. An “amplicon” is an amplified nucleic acid, e.g., a nucleic acid that is produced by amplifying a template nucleic acid by any available amplification method.

“Primer” refers to an oligonucleotide which is capable of acting as a point of initiation of nucleic acid synthesis or replication along a complementary strand when placed under conditions in which synthesis of a complementary strand is catalyzed by a polymerase. Typically, primers are about 10 to 30 nucleotides in length, but longer or shorter sequences can be employed. Primers can be provided in double-stranded form, though the single-stranded form is more typically used. A primer can further contain a detectable label, for example a 5′ end label.

“Probe” refers to an oligonucleotide that is complementary (though not necessarily fully complementary) to a polynucleotide of interest and forms a duplexed structure by hybridization with at least one strand of the polynucleotide of interest. Typically, for PCR probes are oligonucleotides from 10 to 50 nucleotides in length, but longer or shorter sequences can be employed. A probe can further contain a detectable label (e.g., a fluorescent label).

In some embodiments, the probe can be, for example, a guide RNA (gRNA). Typically, gRNAs are short RNA sequences that hybridize to a complementary DNA or RNA sequence and direct a Cas protein to the complementary DNA or RNA sequence to make a break in the DNA or RNA. In the context of the presently disclosed subject matter a gRNA probe can be used, for instance, to target an internal transcribed spacer (ITS) DNA region for pathogen detection or to detect a marker (e.g., a microsatellite) specific for a particular genotype or trait (e.g., fungicide resistance).

As used herein the term “plant tissue” refers to a relatively soft plant tissue, such as, but not limited to, a immature plant tissue, a flower, a seedling, a plant leaf, a tuber, a fruit, or a stem (e.g., a smaller/thinner and/or non-lignified stem). In some embodiments, the plant tissue is any tissue whose outer layer can be punctured by a microneedle having a fracture force of about 1 N or less. In contrast, hard plant tissues, which can include seeds, nuts, grains, roots (e.g., primary roots), larger/harder and/or lignified stems, tree trunks, bark, and other woody materials, are materials whose outer layer is not punctured by a microneedle having a fracture force of about 1 N or less. In some embodiments, the plant tissue is a plant leaf or a portion thereof, a fruit or a stem. In some embodiments, the plant tissue is a plant leaf.

“Animal” as used herein includes humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal can be a transgenic animal. In some embodiments, the term refers to an animal of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), poultry, and horses.

### II. General Considerations

Late blight is caused by the oomycete plant pathogen Phytophthora infestans,⁷ and is one of the major threats to global food security as it adversely affects potato production, one of the world's staple food crops. This oomycete was responsible for the famous Irish potato famine (1845-1849)⁷⁻⁹ and most recently, the late blight pandemic of tomato in eastern USA in 2009.¹⁰⁻¹² Late blight disease spreads rapidly under favorable conditions and can destroy a crop within a few days if left untreated. Every year, the disease causes approximately 6.7 billion USD in crop losses worldwide.¹³ Molecular approaches based on nucleic acid amplification (NAA) are currently the predominant method for diagnosis of late blight, and in particular, for genotyping different strain types of P. infestans with distinct fungicide susceptibility.¹⁴⁻¹⁶ However, current NAA-based diagnostics are limited by not only the cumbersome amplification assay protocols but also the tedious sample preparation steps before the amplification assay. While significant progress has been made in improving the portability of PCR systems to enable lab-on-a-chip detection of plant pathogens,^{17,18} plant sample preparation, namely the extraction of high-quality genomic DNA from plant tissues, remains a major obstacle for performing molecular diagnosis of plant diseases in remote or resource-limited settings.

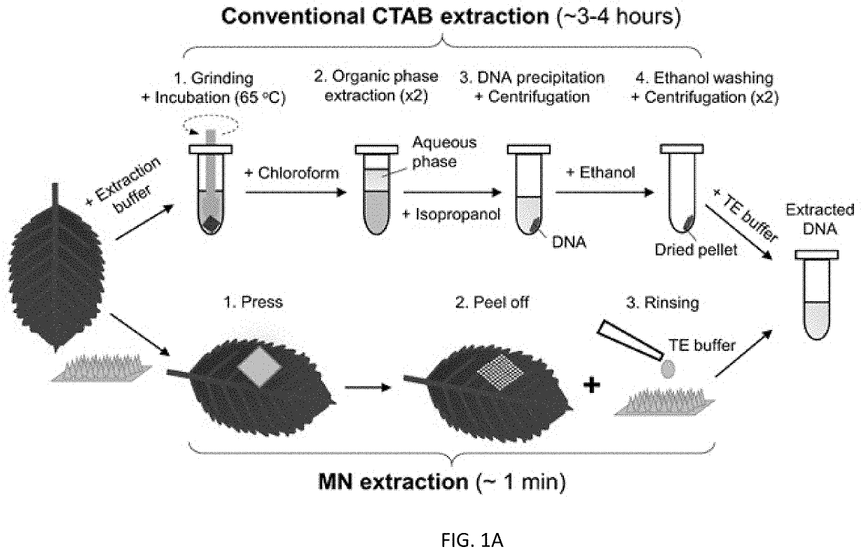

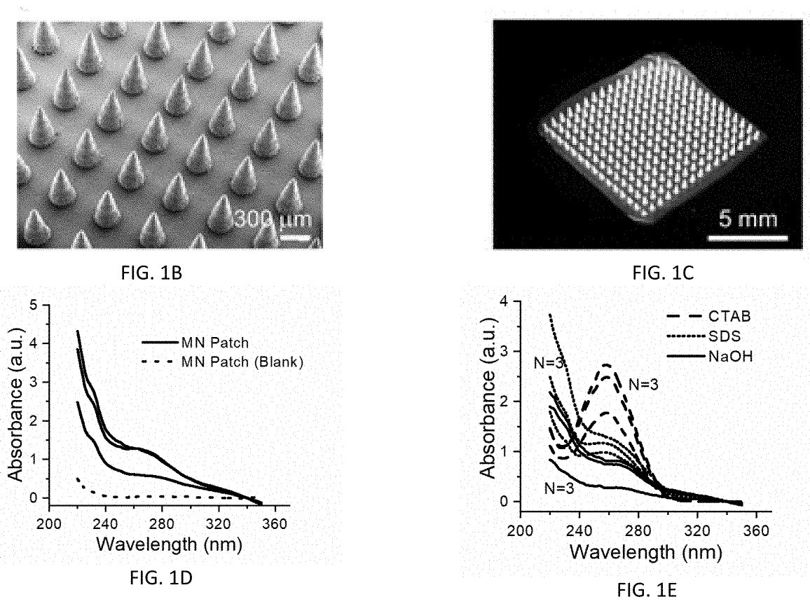

Currently, the most widely used extraction protocol is the cetyltrimethylammonium bromide (CTAB)-based plant DNA extraction protocol,¹⁹ which was developed almost 40 years ago and is still considered a “gold standard” method for isolation of high-quality DNA from the plant tissues. However, CTAB extraction is a complicated and time-consuming process, which involves several steps including: 1) mechanical grinding of plant tissues (e.g., leaves) using mortar and pestle, 2) CTAB-based cell lysis, 3) organic phase DNA extraction, and 4) alcohol-assisted DNA precipitation and purification. See FIG. 1A, top. Alternatively, a NaOH-based quick plant DNA extraction protocol could be used for fast PCR analysis in the laboratory,²⁰ and plastic DNA extraction bags preloaded with extraction buffers are also available for on-site sample preparation via quick grinding for subsequent use with ELISA assays.²¹ FTA cards can also be used to store ground pathogen-infected plant tissue and transport to diagnostic labs for subsequent extraction and DNA testing.^{22,23}

However, development of more rapid, field-applicable, and cost-effective genomic testing platforms is of great interest for farmers and/or extension workers to perform screening assays of plant diseases directly in the field. There are several challenges for developing on-site NAA based plant disease screening platform. One of the challenges is to isolate DNA from infected plant samples without using benchtop equipment. Lab-on-a-chip based DNA extraction technologies developed for mammalian cells or bacteria²⁴⁻²⁷ are difficult to apply to plant cell lysis and DNA isolation due to the presence of rigid plant cell walls. Furthermore, plant leaves, one of the most common sample sources for plant disease screening, consist of plant cells in highly organized sandwich structures, where the epidermal cells and cuticle layers usually protect infected cells. Extraction of genomic DNA from infected plant cells therefore requires breakage through several plant barriers, including cell walls, epidermis, and the waxy cuticle outer layer. Despite the great progress in both laboratory- or chip-based technologies in recent years, none of the methods currently in use provides assay-ready plant DNA in a simple, instrument-free, and field applicable manner. Thus, current methods for diagnosis of plant (and animal) diseases are generally constrained to the laboratory settings due to the cumbersome assay procedures and requirement of bulky equipment.

In some embodiments, the presently disclosed subject matter provides a method of extracting an analyte (e.g., a nucleic acid, a protein, or a small molecule) wherein the extraction method is minimally invasive, does not require cell or tissue lysis, and therefore reduces the sample preparation time from a few hours in a conventional extraction protocol to within one minute. In some embodiments, the presently disclosed subject matter provides an integrated diagnostic platform based on a microneedle MN patch and a smartphone amplification platform for rapid nucleic acid extraction, amplification, and detection directly in the field. In some embodiments, low-cost and disposable MN patches can be used for localized delivery of therapeutic agents such as fungicide to suppress plant pathogen infections with greatly reduced dose of fungicide application. In some embodiments, the presently disclosed methods and systems involve microneedles made of assay-compatible polymers, which isolate substances such as DNA or RNA from plant leaves (or other plant soft tissues or animal tissues) by simple compression and retraction. In some embodiments, the presently disclosed subject matter provides a system comprising a smartphone-based reader which further includes an electric resistor heater-integrated sample cartridge to receive the microneedles and a fluorescent imaging system to detect the assay signals. As described in the Examples hereinbelow, the presently disclosed diagnostic platform can successfully detect both DNA-based (e.g., Phytophthora infestans) and RNA-based (e.g., tomato spotted wilt virus (TSWV)) pathogens from infected plant tissues with minimum operational steps.

### III. Microneedle (MN)-Based Methods

In some embodiments, the presently disclosed subject matter provides a method of extracting an analyte from a plant soft tissue or an animal tissue. Plant soft tissues can include, but are not limited to, immature plant tissue, a flower, a seedling, a plant leaf, a tuber, a fruit, a stem or any other plant tissue that whose outer surface can be punctured by the tip of a microneedle having a fracture force of about 1 newton (N) or less. The method can comprise contacting the plant or animal tissue with a microneedle and removing the microneedle from contact with the plant or animal tissue. Thus, the method can comprise puncturing the tissue with a microneedle and retracting the microneedle. After removal of the microneedle from the plant or animal tissue, the removed microneedle will comprise absorbed analyte (e.g., will be coated with analyte or contain absorbed analyte) extracted from the plant or animal tissue. If desired, the analyte can then be collected by removing it from the microneedle (e.g., by contacting the microneedle with a liquid in which the analyte is soluble). For example, when the analyte comprises one or more nucleic acid, the liquid can be a nucleic acid extraction buffer solution (e.g., Tris-EDTA (TE)) or nuclease-free water.

In some embodiments, the method comprises extracting an analyte from a plant soft tissue. In some embodiments, the method comprises extracting an analyte from a plant leaf, fruit, or stem. In some embodiments, the method comprises extracting an analyte from a plant leaf.

Generally, the microneedle can comprise a solid or hollow body comprising a base at one end and a tip at the opposing end. Typically, the base has a larger diameter than the tip, e.g., such that the body of the microneedle is tapered. In some embodiments, the microneedle has a cone or pyramid shape. The microneedle can be solid, porous, or hollow (i.e., having an opening to an inner cavity at the tip), smooth or rough, dissolvable (e.g., biodegradable) or nondissolvable.

In some embodiments, the microneedle has a fracture force of at least about 0.1 N or at least about 0.2 N. In some embodiments, the microneedle has a fracture force of between about 0.1 N and about 1 N (e.g., about 0.1, 0.2, 0.3, 0.4, 0.5, 0.6, 0.7, 0.8, 0.9 or 1.0 N). In some embodiments, the microneedle has a fracture force of about 1 N.

In some embodiments, the microneedle body comprises, consists essentially of, or consists of a polymer. In some embodiments, the polymer is a hydrophilic polymer or a swellable polymer, e.g., such that the microneedle can absorb fluid (e.g., intracellular water) from the plant or animal tissue. Suitable hydrophilic polymers include, but are not limited to, polyvinyl alcohol (PVA), crosslinked hyaluronic acid (HA), crosslinked polyacrylic acid (PAA), chitosan, or a copolymer thereof. However, the presently disclosed subject matter is not limited to the use of hydrophilic and/or swellable microneedles. In some embodiments, the microneedle can comprise a hydrophobic or amphiphilic polymer. In some embodiments, the microneedle can comprise multiple layers of materials, such as hydrophilic and/or swellable polymer coated hard needles (e.g., metal, silicon, or hydrophobic polymers). In some embodiments, the polymer is porous.

Some polymers, such as chitosan, can be “charge-switchable” such that they are positively charged when exposed to a first pH range (e.g., contacted with an aqueous liquid having a pH in a first pH range) and uncharged when exposed to a second, higher pH range. For instance, chitosan can be positively charged when exposed to an environment having a pH below about 7 and uncharged when exposed to an environment having a pH above about 9. The positive charge of a charged charge-switchable polymer can act like a polyelectrolyte and help to control the binding and releasing of negatively charged analytes, such as nucleic acids, in a pH-dependent manner. The density and isoelectric point of the chitosan-based microneedles and some other polymer microneedles can be tailored by grafting different numbers of carboxylic acid to the polymer. In some embodiments, the use of a charge-switchable polymer can approximately double the amount of nucleic acid extracted from a plant or animal tissue as compared to a non-charge-switchable polymer. Removal of the nucleic acid from a microneedle comprising a charge-switchable polymer can be performed by contacting the microneedle with a liquid at a pH above its isoelectic point.

The type of polymer and amount of polymer crosslinking can affect the fracture force of the microneedle. In addition, the dimensions of the microneedle can affect the fracture force. In some embodiments, the microneedle body has a length of between about 50 microns (μm) and about 1500 μm (e.g., about 50, 100, 150, 200, 300, 400, 500, 600, 700, 800, 900, 1000, 1100, 1200, 1300, 1400, or about 1500 μm). In some embodiments, the microneedle body has a length between about 200 μm to about 1500 μm. In some embodiments, the microneedle has a length of about 800 μm. In some embodiments, the microneedle tip has a diameter between about 1 μm and about 10 μm (e.g., about 1, 2, 3, 4, 5, 6, 7, 8, 9, or about 10 μm). In some embodiments, the microneedle tip has a diameter of about 5 μm. In some embodiments, the diameter of the microneedle base is between about 10 μm and about 500 μm (e.g., about 10, 25, 50, 75, 100, 150, 200, 250, 300, 350, 400, 450, or about 500 μm). In some embodiments, the microneedle base has a diameter between about 100 μm and about 500 μm. In some embodiments, the base diameter is about 150 μm.

The exaction of the analyte can be performed by simple insertion and retraction of the microneedle from the plant or animal tissue, for example, where the microneedle is pressed (e.g., manually or mechanically pressed) to puncture an outer surface of the tissue, such that a portion of the microneedle body comes into contact with inner cells in the tissue. In some embodiments, the contacting comprises placing the tip of the microneedle on an outer surface of the plant tissue (e.g., a cuticle layer of a leaf) or an outer surface of the animal tissue (e.g., a skin surface of an animal, such as a human) and exerting a force on the microneedle sufficient to puncture the outer surface of the plant or animal tissue with the tip of the microneedle, thereby bringing at least a portion of the body of the microneedle into contact with inner cells of the plant tissue or the animal tissue, such as the mesophyll cells of a plant leaf. In some embodiments, the analyte can be absorbed or deposited to the surface of the microneedle and/or into the body of the polymeric microneedle when the microneedle swells by absorbing intracellular water molecules during the extraction process. Thus, in general, the presently disclosed method is free of the use of suction. In addition, the method can be free of cell lysis. Thus, in some embodiments, the analyte can be provided directly for analysis without further purification after removal from the microneedle with a liquid in which the analyte is soluble (e.g., a nucleic acid extraction buffer solution, such as Tris-EDTA, or nuclease-free water). The liquid can be contacted to the microneedle, for instance, by directing a stream of liquid at the microneedle or the microneedle can be dipped in a container holding the liquid. Alternatively, in some embodiments, e.g., when the analyte comprises DNA, the presence of DNA on a microneedle tip can be determined by in situ fluorescent staining with an intercalating dye e.g., that can be contacted to the microneedle after removal of the microneedle from the sample. Additionally or alternatively, in some embodiments, the microneedle can be pre-loaded with reagents for use in the detection and/or analysis of the analyte. For instance, the detection/analysis reagents can be coated on the microneedle body and/or tip or sequestered and/or embedded in pores in the microneedle body and/or tip. The detection/analysis reagents can include, but are not limited to, colorimetric or fluorescent dyes, enzymes, CRISPR reagents, DNA/RNA amplification reagents, and the like. In some embodiments, the extraction can be performed rapidly, e.g., in about 1 minute or less.

The analyte can be any analyte of interest, e.g., any analyte that is expected or suspected of being present in the plant or animal tissue. In some embodiments, the analyte can comprise a native plant or animal nucleic acid (e.g., a plant or animal RNA or DNA) and/or a native plant or animal protein, carbohydrate or other chemical constituent (e.g., a cell wall fragment, etc). Thus, analysis of the analyte can provide genetic and/or physiological information about the plant or animal. In some embodiments, the analyte is a foreign analyte, e.g., comprising a nucleic acid, protein, carbohydrate, lipid, toxin, or other constituent from a pathogen (e.g., a virus, bacteria, fungi or oomycete) or a pest (e.g., an insect, a nematode, a snail, or a slug) that has infected or infested the plant or animal. In some embodiments, the foreign analyte can comprise a drug (e.g., a small molecule and/or synthetic drug), pesticide, or other molecule that was administered to the plant or animal or to which the plant or animal was exposed, or a metabolite thereof.

In some embodiments, e.g., when the analyte is extracted from a plant soft tissue (e.g., a plant leaf), the plant providing the soft tissue can be from a plant cultivated as part of an agricultural crop, such as a crop for use as food for humans or other animals (e.g., livestock), for providing fiber, for providing energy, or for providing an industrial or consumer good. Thus, the plant providing the plant tissue can be selected from the group including, but not limited to, a fruit or vegetable, such as, but not limited to a tomato plant, a potato plant, a pepper plant, a citrus tree, an apple tree, or a banana plant; a soy bean; corn; wheat; cotton; a rubber tree; and tobacco.

In some embodiments, the microneedle can be provided in a patch (e.g., a substantially flat body, such as a polymeric body), from which the microneedle projects. The patch can be provided with a single microneedle or a microneedle array comprising a plurality of microneedles (e.g., 2 to 1,000 microneedles or more). Thus, in some embodiments, contacting the plant tissue or animal tissue with a microneedle comprises contacting the outer surface of the plant tissue or the animal tissue with a plurality of microneedles, wherein the plurality of microneedles is provided in an array format.

The number of microneedles in the patch can be tailored based on the extraction efficiency of the analyte of interest. In some embodiments, the extraction efficiency can be altered based on the type of polymer used to prepare the microneedles (e.g., the hydrophilicity and/or porosity of the polymer used to prepare the microneedles), the size or shape of the individual microneedles, the identity of the analyte of interest, or the type of tissue being extracted. In some embodiments, the patch comprises between 1 microneedle and 1,000 microneedles, inclusive (e.g., 1, 2, 4, 6, 8, 10, 12, 16, 20, 30, 40, 50, 60, 70, 80, 90, 100, 150, 200, 250, 300, 350, 400, 450, 500, 550, 600, 650, 700, 750, 800, 850, 900, 950, or 1000 microneedles).

In some embodiments, the method can further comprise analyzing the analyte, e.g., via immunoassay, gel electrophoresis, sequencing, and/or genotyping. In some embodiments, the analyzing comprises identifying, sequencing and/or quantifying the analyte. In some embodiments, the analysis can be automated, e.g., by the use of a microwell plate with a transparent bottom. For instance, in some embodiments, each microwell in a microwell plate can receive (i) one extract-coated microneedle or a multi-microneedle array/patch, or the collected extract therefrom, and (ii) marker (e.g., loci)-specific reaction mixtures (e.g., containing fluorophore-labeled ssDNA probes that can hybridize with markers of interest). Accordingly, in some embodiments, analysis can be performed “on needle” or “on array”, as well as using collected analyte that has been removed from the microneedle or microneedle array. After the reactions with the analyte, the microwell plate can be scanned by a large field-of-view (FOV) imaging device.

In some embodiments, the presently disclosed subject matter provides a method of detecting a pathogen or pest in a plant or an animal, wherein the method comprises: providing a microneedle or a microneedle patch comprising one or more microneedles, wherein each microneedle has a body with a base and a tip (e.g., where each microneedle body comprises, consists essentially of, or consists of a polymer, optionally a swellable and/or hydrophilic polymer and/or a charge-switchable polymer); contacting the microneedle or microneedle patch with a plant or animal tissue, wherein said plant tissue is a soft tissue (e.g., a plant leaf); removing the microneedle or microneedle patch from contact with the plant or animal tissue, wherein the removed microneedle or one or more microneedle of the removed microneedle patch comprise a foreign analyte extracted from the plant or animal tissue and wherein said foreign analyte is associated with the pathogen or pest; optionally collecting the foreign analyte from the microneedle or microneedle patch; and analyzing the foreign analyte to determine the presence of the pathogen or pest. In some embodiments, the foreign analyte associated with the pathogen or pest comprises a nucleic acid, a protein, a carbohydrate or other chemical constituent or a mixture thereof.

In some embodiments, the contacting comprises placing a tip of the microneedle or of one or more microneedle in the microneedle patch on an outer surface of the plant tissue or the animal tissue (e.g., the cuticle or epidermal layer of a plant soft tissue, such as a plant leaf, or a skin surface of an animal, such as a human or other mammal); and exerting a force on the microneedle or microneedle patch (e.g., manually) sufficient to puncture the outer surface, thereby bringing at least a portion of the body of the microneedle or of one or more microneedle of the microneedle patch into contact with the inner cells of the plant tissue or the animal tissue. In some embodiments, collecting the foreign analyte comprises contacting the microneedle or microneedle patch with a liquid in which the foreign analyte is soluble (e.g., a nucleic acid extraction buffer solution, such as Tris-EDTA, or nuclease-free water), thus dissolving the foreign analyte in the liquid and removing it from the microneedle or microneedle patch and collecting the liquid comprising the dissolved foreign analyte. In some embodiments, the contacting is with between 1 and 1,000 microneedles, inclusive (e.g., 1, 2, 4, 6, 8, 10, 20, 25, 50, 100, 150, 200, 250, 300, 350, 400, 450, 500, 550, 600, 650, 700, 750, 800, 850, 900, 950, or 1,000 microneedles).

The pathogen can be any pathogen of interest, such as a viral pathogen, a fungal pathogen, a bacterial pathogen, or an oomycete pathogen. In some embodiments, the foreign analyte comprises pathogenic DNA or pathogenic RNA. In some embodiments, the pathogen of interest is a viral pathogen and the foreign analyte comprises viral RNA. In some embodiments, the plant pathogen is selected from the group including, but not limited to, Phytophthora infestans, Xanthomonas perforans, Altermaria linariae, and tomato spotted wilt virus (TSWV).

When the foreign analyte comprises or consists of a nucleic acid, the analyzing can comprise nucleic acid amplification. Any suitable method of nucleic acid amplification can be used. For example, the amplification can be performed by a technique selected from polymerase chain reaction (PCR), reverse transcription PCR (RT-PCR), ligase chain reaction (LCR), transcription-based amplification, self-sustained sequence replication (3SR), loop-mediated isothermal amplification (LAMP), reverse transcription LAMP (RT-LAMP), nucleic acid sequence-base amplification (NASBA), rolling circle amplification (RCA), ligation-enabled padlock RCA, CRISPR cassette, or any other suitable technique for nucleic acid amplification. In some embodiments, such as when the method is being performed in a non-laboratory environment, it can be convenient to choose an isothermal amplification technique, e.g., LAMP or RT-LAMP.

Analysis can be performed by any suitable technique. In some embodiments, the analysis can comprise sequencing (e.g., nucleic acid sequencing or amino acid sequencing). Additionally or alternatively, in some embodiments, the analysis can comprise genotyping. In some embodiments, the genotyping can comprise detection of one or more markers of interest selected from, but not limited to, an allele; a locus (e.g., a quantitative trait locus (QTL); a microsatellite (e.g., a short tandem repeat (STR) or a simple sequence repeat (SSR)); a single nucleotide polymorphism (SNP), a single feature polymorphism (SFP), an insertion/deletion polymorphism; a restriction-fragment-length polymorphism (RFLP); an amplified fragment length polymorphism (AFLP) and a CRISPR cassette. In some embodiments, the analyzing is free of cell lysis and/or purification (e.g., nucleic acid purification).

In some embodiments, the presently disclosed subject matter provides a method of genotyping a plant or animal. In some embodiments, the method comprises: providing a microneedle or microneedle patch comprising one or more microneedle, wherein each microneedle has a body with a base and a tip (e.g., where each microneedle body comprises, consists essentially of, or consists of a polymer, optionally a swellable and/or hydrophilic polymer and/or a charge-switchable polymer); contacting the microneedle or microneedle patch with a plant or animal tissue, wherein the plant tissue is a soft tissue (e.g., a plant leaf); removing the microneedle or microneedle patch from contact with the plant or animal tissue, wherein the removed microneedle or one or more microneedle of the removed microneedle patch comprises a native analyte (e.g., a native nucleic acid, protein, carbohydrate or other chemical constituent) extracted from the plant or animal tissue; optionally collecting the native analyte from the microneedle or microneedle patch; and analyzing the native analyte, thereby genotyping the plant or animal.

In some embodiments, the contacting comprises placing a tip of the microneedle or of one or more microneedle of the microneedle patch on an outer surface of the plant tissue or the animal tissue (e.g., the cuticle or epidermal layer of a plant soft tissue, such as a plant leaf, or a skin surface of an animal, such as a human or other mammal); and exerting a force on the microneedle or microneedle patch (e.g., manually) sufficient to puncture the outer surface, thereby brining at least a portion of the body of the microneedle or of one or more microneedle of the microneedle patch into contact with the inner cells of the plant tissue or the animal tissue. In some embodiments, collecting the native analyte comprises contacting the microneedle or microneedle patch with a liquid in which the native analyte is soluble (e.g., a nucleic acid extraction buffer solution, such as Tris-EDTA, or nuclease-free water), thus dissolving the native analyte in the liquid and removing it from the microneedle or microneedle patch and collecting the liquid comprising the dissolved native analyte. In some embodiments, the microneedle patch comprises between 1 and 1,000 microneedles, inclusive (e.g., 1, 2, 4, 6, 8, 10, 20, 25, 50, 100, 150, 200, 250, 300, 350, 400, 450, 500, 550, 600, 650, 700, 750, 800, 850, 900, 950, or 1,000 microneedles).

In some embodiments, the method comprises extracting a native nucleic acid from the plant or animal tissue and the analyzing comprises nucleic acid amplification, such as, but not limited to, a technique selected from PCR, RT-PCR, LCR, transcription-based amplification, 3SR, LAMP, RT-LAMP, RCA, ligation-enabled padlock RCA, NASBA, CRISPR cassette, or any other suitable technique for nucleic acid amplification. In some embodiments, the analyzing comprises or further comprises sequencing and/or genotyping. For example, in some embodiments, the analyzing comprises detection of one or more marker of interest selected from an allele; a locus, optionally a QTL; a microsatellite, optionally a STR or a SSR; a SNP, a SFP, an insertion/deletion polymorphism, a RFLP, and an AFLP. In some embodiments, the analyzing can comprise a multiplexed RCA assay. In some embodiments, the analyzing can comprise Kompetitive allele-specific PCR (KASP) or TaqMan SNP genotyping. In some embodiments, the analyzing can comprise genotyping by sequencing (GBS), MinION sequencing, or CRISPR cassette. In some embodiments, the analyzing is free of purification (e.g., nucleic acid purification) and/or cell lysis.

In some embodiments, the amplification is an isothermal amplification, e.g., eliminating thermal cycling. In some embodiments, the isothermal amplification method is RCA, which uses a circular template to generate single-stranded amplicons with hundreds of thousands of repeated sequences. The products (i.e., the amplicons) can be detected by hybridization with fluorophore-labeled ssDNA probes. For SNP detection, a padlock probe can be used to recognize single-base mismatches in the targets. If the sequences are perfectly matched, ligation of the padlock probe occurs to convert it into an enclosed circular template for the RCA reaction. This approach is analogous to sequencing by ligation (SBL), thereby providing SNP detection accuracy and fidelity.

In some embodiments, the presently disclosed method can be used for rapid analyte extraction and genotyping of plant leaves in order to identify particular desirable traits (e.g., disease, pest or herbicide resistance, drought tolerance, a photosynthetic trait, cold tolerance, etc.) and select progeny in plant breeding. Use of the present microneedles, microneedle arrays, and the patches thereof, to extract analytes can be less invasive than presently used plant tissue sampling techniques, which typically involve removing a portion of the tissue being sampled. In addition, the microneedle patches can be adapted to automate leaf or other tissue sampling, e.g., by the fabrication of microneedle patches (e.g., microneedle array patches) on a strip-roll format. Thus, for example, instead of applying a plurality of individual patches (e.g., square patches), the microneedle patches on the roll can be continuously extended to additional leaves.

In addition to extracting analytes of interest from plant soft tissues and animal tissues, the presently disclosed microneedles, microneedle arrays, and patches thereof can be used to deliver substances of interest to plant soft tissues. Thus, in some embodiments, the presently disclosed subject matter provides a method of delivering a substance of interest to a plant; the method comprising: providing a microneedle or microneedle patch comprising one or more microneedles, wherein each microneedle (e.g., each polymer microneedle) comprises a body comprising a base and a tip, and wherein said body comprises (e.g., is embedded with) or is coated with a substance of interest; and contacting the microneedle or microneedle patch with a plant tissue, wherein said plant tissue is a soft tissue, thereby delivering the substance of interest to the plant. In some embodiments, the plant soft tissue is a plant leaf. Any substance of interest that can be loaded into and/or coated on the microneedles can be delivered. In some embodiments, the substance of interest can be selected from the group including, but not limited to, one or more pesticides, DNA, RNA, one or more proteins, one or more peptides, an antigen (e.g., for stimulating the plant immune system), a vector, a plasmid, a CRISPR cassette, a biological cell, a biological cell component, and/or a vesicle. Thus, the delivery can be used to provide a transgenic or gene edited plant and/or to protect the plant from a pathogen or pest (e.g., an insect or a weed). In some embodiments, the substance of interest comprises a pesticide. For example, the pesticide can be a systemic fungicide, a translaminar fungicide, an herbicide, an insecticide, a biological control agent, or any combination thereof.

In some embodiments, the body of each microneedle comprises, consists essentially of, or consists of a polymer. In some embodiments, the identity of the polymer used to prepare the microneedle or microneedles and/or the size of the microneedle or microneedles and/or the of the microneedles can be tailored to enhance the ability of the microneedle or microneedles to deliver a particular substance of interest, a particular dose of the substance of interest and/or to deliver the substance of interest at a particular rate. In some embodiments, the porosity and/or swellability of the polymer is tailored to provide a desired rate of delivery of the substance of interest. In some embodiments, the polymer is biodegradable and can deliver the substance of interest to the plant tissue at a rate based on the rate of its degradation (e.g., over the course of a one or more minutes, hours, days or weeks).

### IV. Systems

In some embodiments, the presently disclosed subject matter provides a system for detecting an analyte of interest in a biological sample (e.g., a plant soft tissue or an animal tissue). The system can be provided for use in the field, e.g., when it is helpful to detect a pathogen present in a plant crop without having to take samples to a testing/laboratory facility, or in the case of animals, at the point of care (e.g., in a doctor's office, in a veterinarian's office, on a farm, in the home, etc.). In some embodiments, the analyte of interest comprises a nucleic acid native to the source of the biological sample (e.g., the plant soft tissue or animal tissue), a protein native to the source of the biological sample, a nucleic acid from a pathogen or pest (e.g., infecting or infesting the plant or animal), a protein associated with a pathogen or pest, a carbohydrate or other chemical constituent native to the source of the biological sample or from a pathogen or pest, or any combination thereof.

In some embodiments, the system comprises: a microneedle or microneedle patch configured to obtain an analyte extract from the sample; a receiver configured to receive a reaction mixture comprising (i) one or more reagents for detection of an analyte from the sample and (ii) a microneedle, microneedle patch, or an extract from the biological sample obtained using a microneedle or microneedle patch; and a detector for detecting a signal from the reaction mixture associated with a reaction or interaction between an analyte and at least one of the one or more reagents. In some embodiments, the microneedle patch comprises a single microneedle comprising a base and a tip. In some embodiments, the microneedle patch comprises a plurality of microneedles configured in an array, wherein each of the microneedles comprises a body comprising a base and a tip. In some embodiments, the microneedle patch comprises 1 to 1,000 microneedles (e.g., 1, 2, 4, 6, 8, 10, 20, 25, 50, 75, 100, 150, 200, 250, 300, 350, 400, 450, 500, 550, 600, 650, 700, 750, 800, 850, 900, 950, or 1,000 microneedles). In some embodiments, when the microneedle patch comprises a plurality of microneedles (i.e., a microneedle array), the plurality of microneedles are configured in a regular (e.g., square, rectangular, hexagonal, triangular, oval, circular, etc.) or irregular array. In some embodiments, each microneedle body comprises, consists essentially of, or consists of a swellable and/or hydrophilic polymer and/or charge-switchable polymer. In some embodiments, the microneedle or microneedle patch is pre-loaded (e.g., coated or embedded with) one or more reagents for detection and/or analysis of an analyte (e.g., a colorimetric or fluorescent dye, an enzyme, a CRISPR reagent, a DNA/RNA amplification reagent, etc.).

In some embodiments, the biological sample is an animal (e.g., a human or other mammal) and the microneedle patch is configured to obtain an analyte extract from the animal by insertion and removal from a skin surface of the animal. In some embodiments, the animal is a human. In some embodiments, the animal is a non-human mammal, a bird, or a fish. In some embodiments, the biological sample is a soft tissue from a plant and the microneedle patch is configured to obtain an analyte extract from the plant by insertion and removal from the soft tissue of the plant, such as by puncturing a cuticle and/or epidermal layer of the plant soft tissue. In some embodiments, the plant soft tissue is a plant leaf.

In some embodiments, e.g., when the analyte comprises a nucleic acid, the one or more reagents can comprise a polymerase and one or more nucleic acid primers for amplification of a nucleic acid associated with the one or more nucleic acids of interest (e.g., a primer for a particular SNP associated with a particular plant trait of interest or a particular pathogen). When the analyte is expected to contain RNA, the one or more reagents can further comprise a reverse transcriptase. The one or more reagents can further include one or more fluorescent or colorimetric dyes. Suitable dyes include, but are not limited to, hydroxynaphthol blue (HNB) and those sold under the tradenames EVAGREEN™ (Biotium, Inc., Hayward, Calif., United States of America), SYBR™ Green (Molecular Probes, Eugene, Oreg., United States of America), and PICOGREEN™ (Molecular Probes, Eugene, Oreg., United States of America). In some embodiments, the one or more fluorescent or colorimetric dyes comprise HNB.

In some embodiments, the system can further comprise an attachment configured to position the receiver with respect to the detector (e.g., to align the receiver with a camera lens or a light detector). In some embodiments, the attachment can also include a grip so that the detector and receiver can be handheld.

In some embodiments, the detector comprises a camera configured to capture an image of the receiver or a portion thereof. For example, in some embodiments, the detector can be a consumer electronics device that includes a camera and the camera can be configured to capture an image of the receiver. The consumer electronics device can be, for example, a smartphone or a tablet.

In some embodiments, the consumer electronics device can be configured to display data related to a signal obtained by the camera and/or to transfer the data (e.g., wirelessly) to another device (e.g., a laptop). In some embodiments, the detector can further comprise a lens, a diffuser, or a combination thereof. In some embodiments, the detector can comprise one or more light sources (e.g., LEDs or laser diodes) configured to illuminate the receiver. In some embodiments, the receiver comprises a chamber or chip. In some embodiments, the chamber or chip is polydimethylsiloxane (PDMS) or another suitable polymer that is non-reactive to any reactions performed in the receiver.

In some embodiments, e.g., when thermocycling is performed as part of a nucleic acid amplification process, the system further comprises a heating device configured to heat the receiver to a pre-determined temperature or temperatures for a pre-determined period of time or times. The heating device can be, for example, a flexible polyamide heating device that can be powered by batteries, or a self-heating material (e.g., an Mg—Fe alloy in the presence of water).

In some embodiments, the system can comprise one or more batteries. For example, a battery (solar or electric) can be used to provide power to heat the receiver or to power one or more light sources associated with the detector.

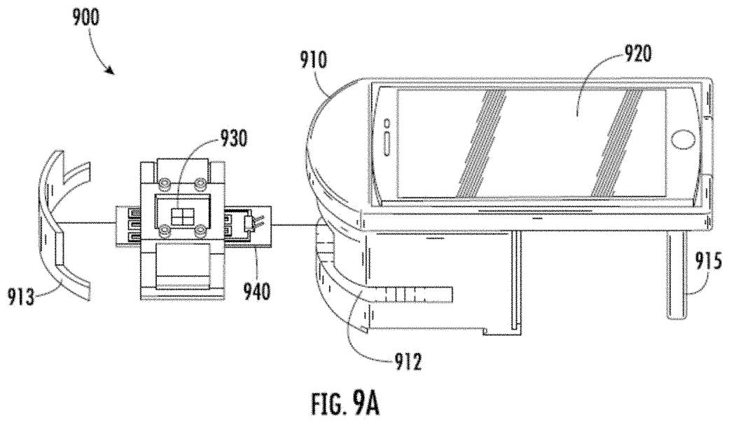

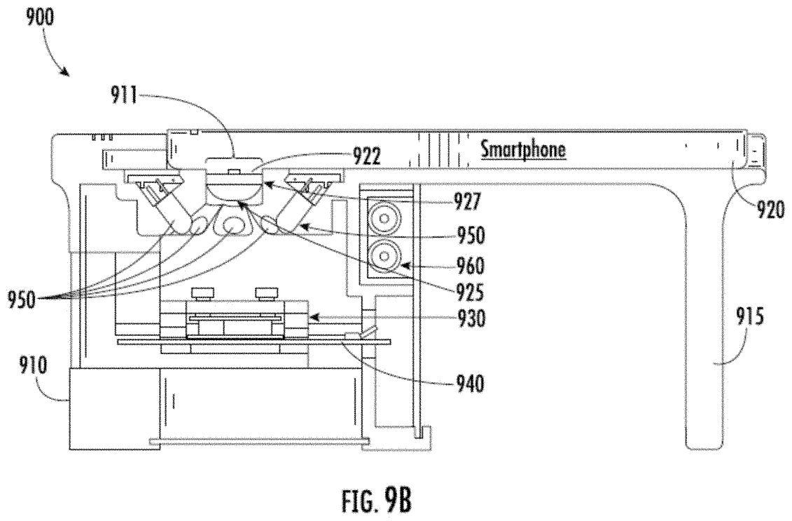

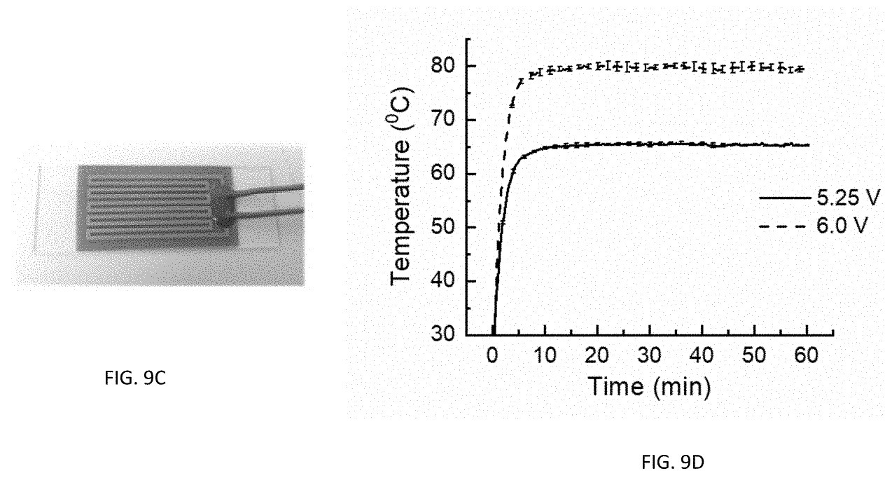

An exemplary attachment 910 for system 900 of the presently disclosed subject matter is shown in FIGS. 9A and 9B. For example, FIG. 9A shows a partial side view of attachment 910 configured to position receiver 930 with respect to detector 920 (i.e., a smartphone). Attachment 910 can comprise a rigid plastic body and includes slot 912 into which receiver 930 can be inserted (i.e., when door 913 is removed). In FIG. 9A, receiver 930 is a LAMP cassette and is shown to removed and to the left side of attachment 910 to provide a better view. Under receiver 930 is heating device 940 (i.e., a flexible polyamide heater). In FIGS. 9A and 9B, dectector 920 is shown as a smartphone and is shown placed on the top of attachment 910, which is configured to hold detector 920 in place with camera 922 positioned in opening 911 in the body of attachment 910 so that camera 922 from detector 920 is positioned to obtain a signal from receiver 930. FIG. 9B shows attachment 910 with receiver 930 in place in the body of attachment 910 and positioned under camera 922, with heating device 940 under receiver 930. LED lights 950 are positioned to illuminate receiver 930. Emission filter/diffuser 927 and lens 925 are positioned directly adjacent to opening 911 for camera 922, i.e., between camera 922 and receiver 930. A compartment for batteries 960 to power heating device 940 is shown in the body of attachment 910 in FIG. 9B. Attachment 910 further comprises handle/grip 915.

In some embodiments, the presently disclosed subject matter provides a method of detecting a presence and/or an amount of one or more analyte of interest in a biological sample wherein the method comprises use of the presently disclosed system. For example, the method can comprise providing the presently disclosed system, contacting the microneedle or microneedle patch with the biological sample, removing the microneedle or microneedle patch from the biological sample, and (i) placing the microneedle or microneedle patch in the receiver or (ii) rinsing the microneedle or microneedle patch with a liquid in which the analyte of interest is soluble, collecting the liquid following the rinsing (when it contains dissolved analyte from the needle or patch), and introducing the collected liquid or a portion thereof into the receiver. In some embodiments, the analyte of interest is associated with a pathogen or pest. For example, in some embodiments, the analyte of interest is a nucleic acid from a pathogen or pest.

### V. Nucleic Acid Techniques

In some embodiments, it is desirable as part of one of the presently disclosed methods or as part of the use of the presently disclosed system to amplify a nucleic acid sequence. Nucleic acid amplification can be performed using any of several nucleic acid amplification procedures that are well known in the art and including those described hereinabove, e.g., PCR, RT-PCR, LCR, 3SR, LAMP, RT-LAMP, NASBA, RCA, CRISPR cassette, etc. Generally, nucleic acid amplification is the chemical or enzymatic synthesis of nucleic acid copies that contain a sequence complementary to a nucleic acid sequence being amplified (template). The methods and systems of the presently disclosed subject matter can use any nucleic acid amplification or detection methods known to one skilled in the art, such as those described in U.S. Pat. No. 4,683,195 (Mullis), U.S. Pat. No. 5,130,238 (Malek), U.S. Pat. No. 5,525,462 (Takarada et al.); U.S. Pat. No. 6,114,117 (Hepp et al.); U.S. Pat. No. 6,344,317 (Urnovitz); U.S. Pat. No. 6,448,001 (Oku); U.S. Pat. No. 6,528,632 (Catanzariti et al.); and PCT Pub. No. WO 2005/111209 (Nakajima et al.); all of which are incorporated herein by reference in their entirety.

The PCR process is well known in the art and is thus not described in detail herein. For a review of PCR methods and protocols, see, e.g., Innis et al., eds., PCR Protocols, A Guide to Methods and Application, Academic Press, Inc., San Diego, Calif. 1990; and U.S. Pat. No. 4,683,202 (Mullis); which is incorporated herein by reference in its entirety. PCR reagents and protocols are also available from commercial vendors, such as Roche Molecular Systems (Pleasanton, Calif., United States of America), Thermo Fisher Scientific (Waltham, Mass., United States of America) and New England Biolabs (Ipswich, Mass., United States of America). In some embodiments, PCR is carried out as an automated process with a thermostable enzyme. In this process, the temperature of the reaction mixture is cycled through a denaturing region, a primer annealing region, and an extension reaction region automatically. Machines specifically adapted for this purpose are commercially available.

In some embodiments, reverse transcribed or amplified nucleic acids can be modified nucleic acids. Modified nucleic acids can include nucleotide analogs, and in certain embodiments include a detectable label and/or a capture agent. Examples of detectable labels include, without limitation, fluorophores, radioisotopes, colorimetric agents, light emitting agents, chemiluminescent agents, light scattering agents, enzymes and the like. Examples of capture agents include, without limitation, an agent from a binding pair selected from antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B 12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) pairs, and the like. Modified nucleic acids having a capture agent can be immobilized to a solid support in certain embodiments.

In some embodiments, the presently disclosed subject matter provides a method of sequencing and/or amplification that comprises selectively hybridizing a probe nucleic acid to a nucleic acid target. The probe is generally an oligonucleotide or polynucleotide that can selectively hybridize to at least a portion of a target nucleic acid sequence under conditions that allow for or promote selective hybridization. In general, a probe can be complementary to the coding or sense (+) strand of DNA or complementary to the non-coding or anti-sense (−) strand of DNA (sometimes referred to as “reverse-complementary”). Probes can vary significantly in length. A length of about 10 to about 100 nucleotides, such as about 15 to about 75 nucleotides, e.g., about 15 to about 50 nucleotides, can be preferred in some applications such as PCR, whereas a length of about 50 to about 1×10⁶ nucleotides can be preferred for chromosomal probes and a length of about 5,000 to about 800,000 nucleotides or more preferably about 100,000 to about 400,000 for BAC probes. “Probe” can also refer to the use of a short oligonucleotide that may contain a reporter molecule, such as but not limited to a probe sold under the tradename TAQMAN™ (Roche Molecular Systems, Pleasanton, Calif., United States of America), capable of being used to detect and quantify the abundance of an amplicon.

Hybridization techniques are well known in the art and are described by Sambrook, J., E. F. Fritsch, and T. Maniatis (Molecular Cloning: A Laboratory Manual, Cold Spring Harbor Laboratory Press, Cold Spring Harbor, N.Y., chapters 9 and 11, (1989)) and Current Protocols in Molecular Biology (F. M. Ausubel et al., eds., John Wiley & Sons, Inc., sections 2.10 and 6.3-6.4 (1995)). Moderately stringent conditions for filter hybridizations include hybridization in about 50% formamide, 6×SSC at a temperature from about 42° C. to 55° C. and washing at about 60° C. in 0.5×SSC, 0.1% SDS. Highly stringent conditions are defined as hybridization conditions as above, but with washing at approximately 68° C. in 0.2×SSC, 0.1% SDS. SSPE (1×SSPE is 0.15 M NaCl, 10 mM NaH₂PO₄, and 1.26 mM EDTA, pH 7.4) can be substituted for SSC (1×SSC is 0.15 M NaCl and 15 mM sodium citrate) in the hybridization and wash buffers; washes, optionally at least two washes, are performed for 15 minutes after hybridization is complete.

It should be understood that the wash temperature and wash salt concentration can be adjusted as necessary to achieve a desired degree of stringency by applying the basic principles that govern hybridization reactions and duplex stability, as known to those skilled in the art and described further below (see e.g., Sambrook et al., supra). When nucleic acids of known sequence are hybridized, the hybrid length can be determined by aligning the sequences of the nucleic acids (for example, using BLAST or a variant) and identifying the region or regions of optimal sequence complementarity. The hybridization temperature for hybrids anticipated to be less than 50 base pairs in length should be 5 to 10° C. less than the melting temperature (Tₘ) of the hybrid, where Tₘ is determined according to the following equations. For hybrids less than 18 base pairs in length, Tₘ (degrees C.)=2(# of A+T bases)+4(# of G+C bases). For hybrids greater than 18 base pairs in length, Tₘ (degrees C.)=81.5+16.6 (log₁₀ [Na+])+0.41 (% G+C)−(600 N), where N is the number of bases in the hybrid, and [Na+] is the concentration of sodium ions in the hybridization buffer.

In some embodiments, nucleotide sequencing can be by solid phase single nucleotide sequencing methods and processes. Solid phase single nucleotide sequencing methods involve contacting sample nucleic acids and a solid support under conditions in which a single molecule of sample nucleic acid hybridizes to a single molecule of a solid support. Such conditions can include providing the solid support molecules and a single molecule of sample nucleic acid in a “microreactor.” Such conditions also can include providing a mixture in which the sample nucleic acid molecule can hybridize to solid phase nucleic acid on the solid support.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### DNA Extraction and Amplification by Conventional PCR

Fabrication of MN Patches:

Patches were fabricated using laser ablation-fabricated polydimethylsiloxane (PDMS) molds (Blueacre Technology Ltd., Dundalk, Ireland). The dimensions of each mold were approximately 10 mm×10 mm, with 15×15 arrays of microneedle conical cavities with a height of 800 micrometers (μm). 0.5 mL of a solution of poly(vinyl alcohol) (PVA, 31-50 kDa, 10 wt %) was added to each mold and the molds were placed in a vacuum (100 kPa) chamber for 10 minutes to draw the PVA solution into the cavities and achieve the desired viscosity. The molds were kept overnight at 25° C. in a chemical hood vacuum. After drying, the patches were separated from the molds and stored at 25° C. in a sealed Petri dish.

MN Patch-Based DNA Extraction:

DNA was extracted in two steps from a fresh plant leaf. First, a MN patch was manually pressed gently onto the leaf surface. The MN patch was then removed and rinsed using 100 μL of TE buffer (10 mM Tris-HCl, 0.1 mM EDTA, pH 8.0) for collecting absorbed DNA from the needle tips. A new MN patch was used for each extraction and the concentrations of extracted DNA in the 100 μL rinse buffer was measured using a UV-Vis spectrophotometer (sold under the tradename NANODROP™ One Microvolume UV-Vis Spectrophotometer, Thermo Fisher Scientific, Waltham, Mass., United States of America). For PCR reactions, 1 μL or 2 μL of MN-extracted solutions was used without further purification.

CTAB-Based DNA Extraction:

Leaf samples were placed in 1.5 mL microcentrifuge tubes and homogenized using disposable pestles after the addition of 150 μL of extraction buffer (0.35 M sorbitol, 0.1 M Tris, 0.005 M EDTA, 0.02 M sodium bisulfite, pH 7.5). Then, 150 μL of nuclei lysis buffer (0.2 M Tris, 0.05 M EDTA, 2.0 M NaCl, and 2% hexadecyltrimethylammonium bromide, pH 7.5) and 60 μL of 5% N-lauryl sarcosine were added to the homogenized solution and vortexed to mix. The microcentrifuge tubes were incubated in a water bath at 65° C. for 30 minutes and mixed with one volume of chloroform/isoamyl alcohol (24:1). After chloroform extraction, the mixture was centrifuged at 12,000 rpm for 15 minutes. The aqueous phase containing DNA was transferred to new centrifuge tubes and mixed with one volume chloroform/isoamyl alcohol (24:1) to repeat the chloroform extraction. Then, one volume of cold 100% isopropyl alcohol and 0.1 volume of 3 M sodium acetate (pH 8.0) were mixed with the aqueous phase of each sample and kept overnight at −20° C. for DNA precipitation. Samples were then centrifuged at 13,000 rpm for 5 minutes. The supernatants were discarded, and the precipitated DNA pellets were washed twice using 1 mL cold 70% ethanol and air-dried in a hood for 30 minutes. Dry pellets were resuspended in 50 μL of TE buffer (10 mM Tris-HCl, 0.1 mM EDTA, pH 8.0). Concentrations of the extracted DNA were measured by UV-Vis spectroscopy and diluted to about 10 nanograms per μL for PCR.

Leaf Inoculation:

Leaves were inoculated using P. infestans, a US-23 genotype, isolate NC 14-1. To collect P. infestans sporangia, infected leaves were held vertically, and 1 mL of DI water was sprayed on the abaxial side of the leaves. The runoff liquid containing sporangia was collected in a beaker and the number of sporangia/mL in the collected solution was estimated using a hemocytometer (Hausser Scientific, Horsham, Pa., United States of America). The solution was then diluted to 10,000 sporangia/mL for inoculation onto healthy plants. For spraying, the diluted solution was transferred in a 15 mL tube with a spray cap (Container & Packaging Supply, Inc., Eagle, Id., United States of America). Healthy leaves were inoculated in a 1.5% water agar plate. Healthy leaves facing the abaxial side up were placed on the non-agar side of the plate and sprayed with 2 mL of sporangia solution on each leaf. The agar sides of the plate were placed on top of the non-agar sides and the plates were sealed using parafilm and incubated at room temperature. For negative controls, healthy leaves were sprayed with 2 mL of DI water and kept under the same conditions as the inoculated leaves. After 3-4 days, hyphae typically come out on the surface as a white growth and generate new sporangia. Leaves also present lesions (dark spots).

Conventional PCR Amplification:

Reagents were purchased from Thermo Fisher Scientific (Waltham, Mass., United States of America). PCR reactions were run on a thermal cycler (sold under the tradename SIMPLIAMP™, Life Technologies Corporation, Carlsbad, Calif., United States of America). For rbcL gene amplification of plant plastid DNA, Rbcla-F (5′-ATGTCACCACAAACAGAGACTAAAGC-3′ (SEQ ID NO: 1), and rbcLajf634R (5′-GAAACGGTCTCTCCAACGCAT-3′ (SEQ ID NO: 2)) primers were used. These primers generate an amplicon of 670 basepairs (bp). The reaction master mix for rbcL gene detection was 1×PCR buffer, 0.1 mM dNTPs, 0.4 μM for each primer, 1.8 mM magnesium chloride, 0.1 mg/mL bovine serum albumin (BSA), and 0.04 U/μL Taq DNA polymerase. The amplification reactions were performed in 25 μL volumes, and for each PCR reaction, an about 10 ng template DNA was used. For negative controls, no DNA was used in the amplification reaction. Cycling conditions were 94° C. for 2 minutes (initial denaturation) followed by 35 cycles of 30 seconds at 94° C. (denaturation), 45 seconds at 54° C. (annealing), and 45 seconds at 72° C. (extension). Then, the temperature was set to 72° C. for 5 minutes for final extension followed by a hold at 4° C. For late blight disease, two P. infestans-specific primers PINF2 (5′-CTCGCTACAATAGCAGCGTC-3′; SEQ ID NO: 3) and ITS5 (5′-GGAAGTAAAAGTCGTAACAAGG-3′; SEQ ID NO: 4) were used for the amplification reaction. The reaction master mix was 1×PCR buffer, 0.1 mM dNTPs, 0.4 μM of each primer, 1.8 mM magnesium chloride, 0.1 mg/mL BSA, and 0.02 U/μL Taq DNA polymerase. The amplification reactions were performed in 25 μL volumes by mixing 24 μL of the master mix and 1 μL of template DNA (concentration about 10 ng/μL). Thermal cycling conditions were 94° C. for 2 minutes (initial denaturation) followed by 35 cycles of 15 seconds at 94° C. (denaturation), 15 seconds at 56° C. (annealing), and 15 seconds at 72° C. (extension). Then, the temperature was set to 72° C. for 5 minutes for final extension followed by a hold at 4° C. DNA extracted from healthy leaves, or no DNA (sample from blank MN patch), were used as negative controls.

Gel Electrophoresis: After amplification, gel electrophoresis was performed to visualize amplified PCR products. Agarose SYBR™ safe DNA gel stain (20000×), 10× Tris-borate-EDTA (TBE) buffer, DNA gel loading dye, and 100 bp DNA ladder were purchased from Thermo Fisher Scientific (Waltham, Mass., United States of America). PCR-amplified products were visualized in 2% agarose gel. The agarose gel was prepared by mixing 1.2 g of agarose and 6 μL of SYBR™ safe DNA gel stain with 60 mL of 1×TBE buffer in a glass bottle, which was microwaved for 1.5 minutes. The microwave was stopped every 30 seconds to suspend undissolved agarose by gentle swirling of the glass bottle. The dissolved agarose solution was kept at room temperature to cool to 60° C. before being poured into a gel casting tray. After solidification, the gel was transferred to a Sub-Cell GT agarose gel electrophoresis system (Bio-Rad Laboratories, Hercules, Calif., United States of America). 1×TBE buffer was used to run the agarose gel, and 5 μL of PCR-amplified product and 1 μL of 6×DNA loading dye were mixed for gel loading. After the gel was run, the image was captured using an imaging system sold under the tradename E-Gel® with Blue Light Base (Thermo Fisher Scientific, Waltham, Mass., United States of America).

Quantitative PCR (qPCR) Amplification:

For a real-time assay of late blight disease detection, a dye sold under the tradename EVAGREEN™ (20,000×) was purchased from Biotium Inc. (Hayward, Calif., United States of America). The dye was diluted to 20× in DI water prior to addition to a master PCR mix. Other agents and primers were purchased from Thermo Fisher Scientific (Waltham, Mass., United States of America).

For real-time detection, two primers PINF2 (SEQ ID NO: 3) and HERB2 (5′-CGGACCGACTGCGAGTCC-3′; SEQ ID NO: 5) were used. These primers amplify a 100 bp region of the internal transcribed spacer (ITS) region 2 of P. infestans. The amplification reactions were run in 25 μL volumes comprising 1 μL of template DNA (concentration about 1 ng/μL), 2.5 μL of 10×PCR buffer, 1.25 μL of dNTPs (2 mM each), 1 μL of 10 μM PINF2, 1 μL of 10 μM HERB2, 1.25 μL of 20× dye, 1.25 μL of 50 mM magnesium chloride, 0.05 μL of 50 mg/mL BSA, 0.1 μL of 5 U/μL Taq DNA polymerase, and 15.6 μL of DI water. Thermal cycling conditions were 94° C. for 2 minutes (initial denaturation) followed by 35 cycles of 15 seconds at 94° C. (denaturation), 15 seconds at 56° C. (annealing), 15 seconds at 72° C. (extension), and the fluorescence signal was captured for each cycle after the extension stage. After thermal cycling, the temperature was maintained at 72° C. for 5 minutes for the final extension of products. Melt curve analyses were performed by slowly increasing the temperature to 95° C. to find the melt temperatures of the amplified product.

Discussion:

MN patches made of polyvinyl alcohol (PVA) were fabricated through a simple vacuum-based micromolding procedure.³⁰ Each patch consists of a 15×15 microneedle array, and each needle is 800 μm in height, 150 μm in base radius, and 5 μm in tip radius. See FIGS. 1B and 1C. The fracture force of polymeric MN patch can be up to ˜1 N/needle,^{31,32} strong enough to insert into skin and plant tissues without breaking.³³⁻³⁶ In a typical MN extraction protocol, a fresh MN patch is gently placed on the surface of the leaf of interest. Then, a punctuation force is delivered to the patch by finger pressing for a few seconds; next, the patch is peeled off and rinsed with 100 microliters (μL) of tris(hydroxymethyl)aminomethane-ethylenediaminetetraacetic acid (TE) buffer. See FIG. 1A, bottom. The entire process takes less than 1 min, orders of magnitude faster and simpler than the conventional extraction protocol that usually takes ˜3-4 hours minimally. See FIG. 1A, top. The MN patch performs two roles during the process: 1) it penetrates deep into plant tissue in a minimally invasive fashion to break hard-to-lyse plant cell walls and release encapsulated nucleic acid materials; 2) it absorbs and concentrates DNA molecules on the surface of MN tips during the needle retraction. After MN retraction, plant DNA can be released from the needle tips by rinsing with elution buffer. See FIG. 1A, bottom. The extracted samples are subsequently used for nanodrop measurement and PCR assay without further purification or stored at 4° C. for future use.

FIGS. 1D and 1E present representative UV absorption spectra of genomic DNA samples extracted from fresh tomato leaves using different DNA extraction methods. The characteristic UV absorption at 260 nm (A260) estimates the concentration of total DNA in the extraction solution, while the ratios of A260/A280 and A260/A230 measure the purity of DNA against proteins and polysaccharides, respectively.³⁷ From the spectral data, it is clear that the MN patch extracted a significant amount of DNA from plant leaves based on the appearance of A260 absorption for all samples tested. See FIG. 1D. In contrast, the solution from the blank MN patch did not show any significant absorption at the 260 nm. See FIG. 1D. The purity of MN-extracted DNA samples was compared with those obtained by conventional extraction methods, including CTAB extraction,¹⁹ sodium dodecyl sulfate (SDS) extraction,³⁸ and NaOH rapid extraction.²⁰ See FIG. 1E and Table 1, below. Among the three conventional protocols, only the NaOH method does not include a purification step. For pure DNA, the standard values of A260/A280 and A260/A230 are expected to be between 1.8-2.0 and >1.8, respectively.³⁷ DNA isolated by the CTAB method demonstrated the closest UV-Vis readings to the target ranges. See Table 1, below. On the other hand, the A260/A230 ratio (DNA/polysaccharide) of DNA extracted by the MN patch was quite similar to the results of SDS and NaOH methods, suggesting a comparable DNA quality via a much simpler procedure.

TABLE 1
UV-Vis readings of DNA solutions extracted by different methods.

| | A260/A280 | A260/A230 |
|---|---|---|
| Target range | 1.8-2.0 | >1.8 |
| CTAB extraction | 2.14 ± 0.01 | 2.24 ± 0.23 |
| SDS extraction | 1.64 ± 0.09 | 0.63 ± 0.17 |
| NaOH extraction | 1.40 ± 0.05 | 0.48 ± 0.02 |
| MN extraction | 1.22 ± 0.04 | 0.52 ± 0.02 |

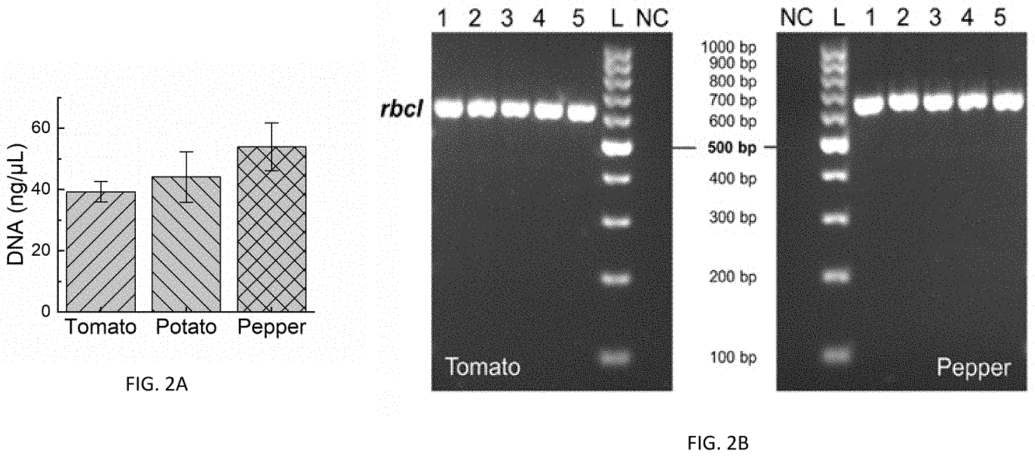

In the presently disclosed MN extraction approach, DNA samples were extracted from fresh leaves without using any chemicals, and as a result, the DNA was directly amplifiable without any purification. To demonstrate that, DNA was extracted by MN patches from fresh tomato, potato, and pepper leaves. See FIG. 2A. Five different leaves for each species were tested, and each time a new MN patch was used. After extraction, PCR amplification reactions were performed using 1 μL of needle-extracted samples to amplify the ribulose-bisphosphate carboxylase gene (rbcL) of plant plastid DNA. Next, gel electrophoresis was performed to visualize the amplified DNA bands. The MN-extracted samples were successfully amplified by the PCR reaction and the characteristic bands at around 670 bp³⁹ were observed for all samples tested from tomato and pepper species. See FIG. 2B. The results confirm that the MN-extracted DNA is directly applicable for PCR amplification without the need of further purification.

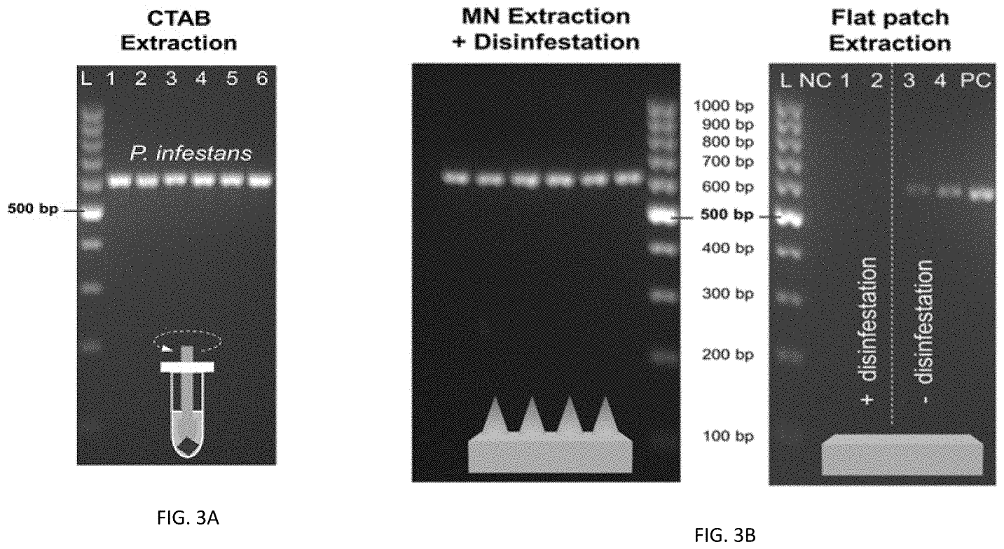

Next, the MN patch-based DNA extraction method was tested to extract much less abundant plant pathogen DNA from laboratory-inoculated plant leaves. Fresh tomato leaves were collected from P. infestans-inoculated tomato plants. Four days after inoculation, the infected leaves were subjected to both MN and CTAB extraction. PCR amplifications were then performed using 1 μL of extracted DNA samples and the results were characterized by gel electrophoresis. See FIGS. 3A and 3B. To rule out the possibility that MN extraction only collects pathogen DNA from the surface of leaf samples, all leaves used in MN extraction were pre-disinfested by rinsing with a 5% bleach solution for a few seconds followed by rinsing with DI water twice. The gel electrophoresis results show that the characteristic band at around 610 bp⁴⁰ was observed for all CTAB (see FIG. 3A) and MN-extracted samples (see FIG. 3B, left), suggesting a successful detection of P. infestans in both methods from laboratory-inoculated samples.

To further validate that MN patches indeed extract in-planta pathogen DNA, flat PVA patches (no sharp microneedles) were used as a control. See FIG. 3B, right. When a similar surface disinfestation procedure was applied to the flat patch extraction, no P. infestans bands were detected by flat patches as indicated by the gel electrophoresis. See FIG. 3B, right, lane 1-2. In contrast, without performing surface disinfestation, flat patches also detected the presence of P. infestans from the surface of infected samples. See FIG. 3B, right, lane 3-4. These results suggest that in order to probe pathogen DNA inside plant tissues, microneedle structures are required in order to break the leaf surface and penetrate into deep tissues.

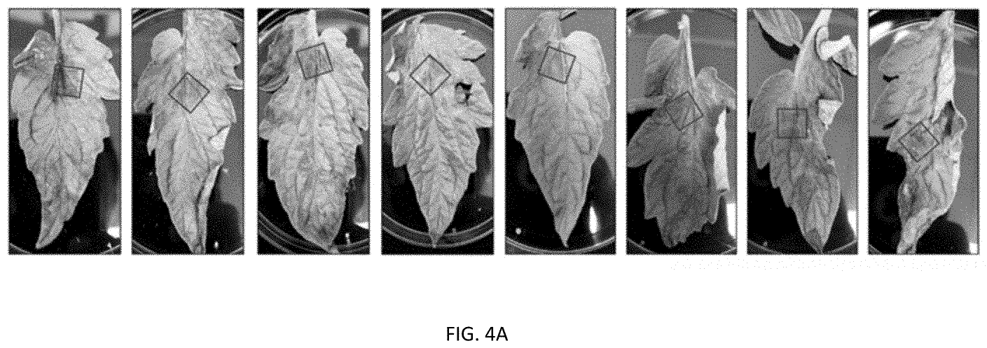

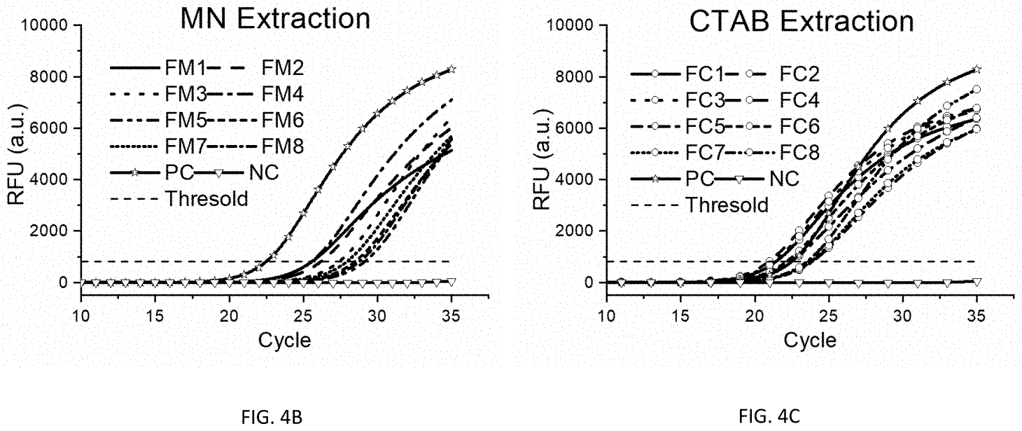

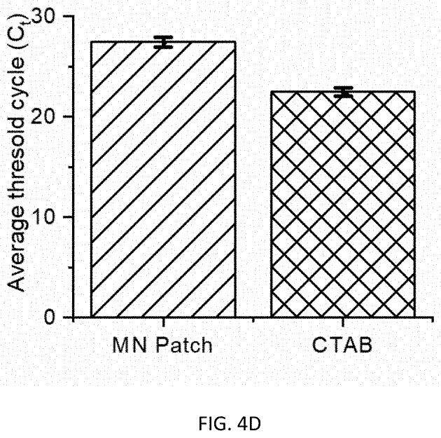

Finally, the MN extraction method was tested to detect late blight disease in field samples. See FIGS. 4A-4D. Eight infected tomato leaves were collected. See FIG. 4A. DNA were extracted from these samples first by the MN patch and then the CTAB method as usual. After extraction, real-time PCR amplifications were carried out for disease detection. See FIGS. 4B and 4C. The presence of P. infestans was detected by both methods in all field samples. The threshold values of the MN method were ˜5 cycles higher than the CTAB method for the field samples. See FIG. 4D.

### Example 2

### DNA Extraction and Isothermal Amplification by a Smartphone Device

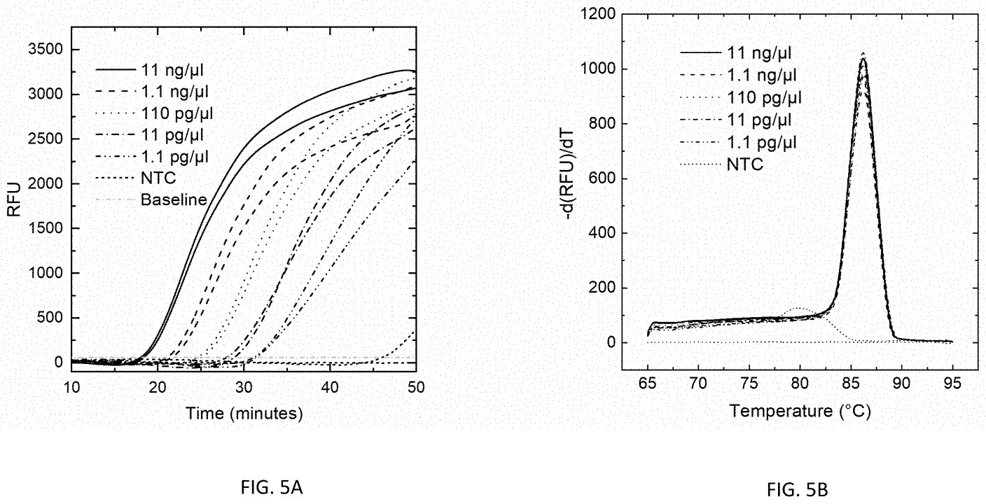

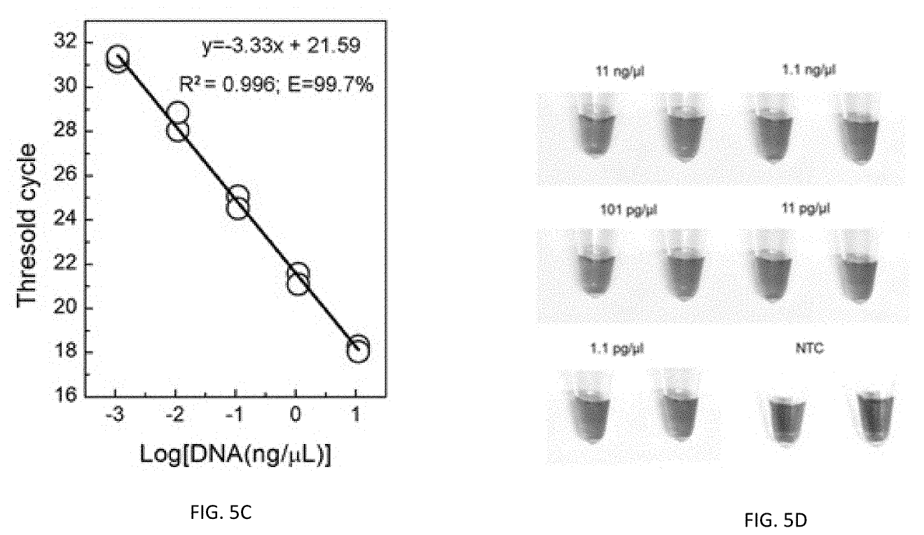

In Example 1, it was demonstrated that MN-extracted DNA is directly applicable for conventional PCR amplification. Here, it is demonstrated that the extracted DNA can also be amplified by an isothermal assay method, namely loop-mediated isothermal amplification (LAMP). LAMP offers several advantages over PCR, such as no need for thermal cycling, higher sensitivity and specificity, and therefore is more suited for in-field applications. For P. infestans detection, a new set of LAMP primers was designed based on the internal transcribed spacer (ITS) region of the pathogen. See Table 2, below. The performance of the P. infestans-specific LAMP assay was optimized to minimize false-positive amplifications by implementing a strategy to include a chemical additive, hydroxynaphthol blue (HNB), in parallel with intercalating dyes such as that sold under the tradename EVAGREEN™ (Biotium, Inc., Hayward in the “one-pot” fluorescent LAMP reaction. HNB interacts with intercalating dyes, increasing their temperature stability and decreasing interference with the amplification process, and therefore allowing for greater fold changes in fluorescent assay signal and fewer false-positive reactions. In addition, the combination of HNB and intercalating dye allows for dual assay signal readout in both fluorometric and colorimetric modes. Finally, the performance of the new LAMP assay was validated by using twenty-three isolates of P. infestans. The developed LAMP assay is specific for P. infestans and does not amplify other Phytophthora species or other fungal and bacterial pathogens that infect potato and tomato.²⁸ The detection limit of the developed real-time LAMP assay is 1 pg/μl, (see FIGS. 5A-5D), which is 10 times lower than PCR.

TABLE 2
LAMP primers.

| Primer | | |
|---|---|---|
| Name | Primer sequence (5′-3′) | Size |
| F3 | CTCCAAAAGTGGTGGCATTG | 20 |
| | (SEQ ID NO: 6) | |
| B3 | GCAACAGCAAAGCCGATTC | 19 |
| | (SEQ ID NO: 7) | |
| FIP | TCTCCATTAACGCCGCAGCAGTGGACGCTGCTATT | 40 |
| | GTAGC (SEQ ID NO: 8) | |
| BIP | CGTGGTATGGTTGGCTTCGGCATGGTTCACCAGTC | 41 |
| | CATCAC (SEQ ID NO: 9) | |
| LoopF | ACAAACCGGTCGCCAACTC | 19 |
| | (SEQ ID NO: 10) | |
| LoopB | ATGCGCTTATTGGGTGATTTTCCTG | 25 |
| | (SEQ ID NO: 11) | |

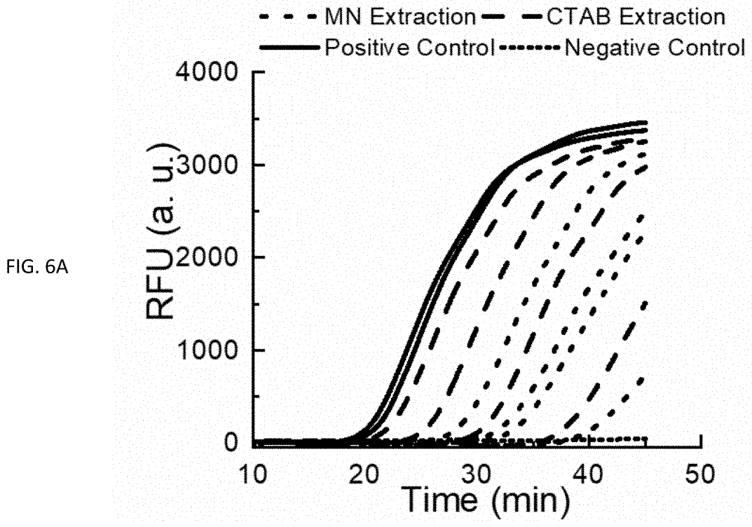

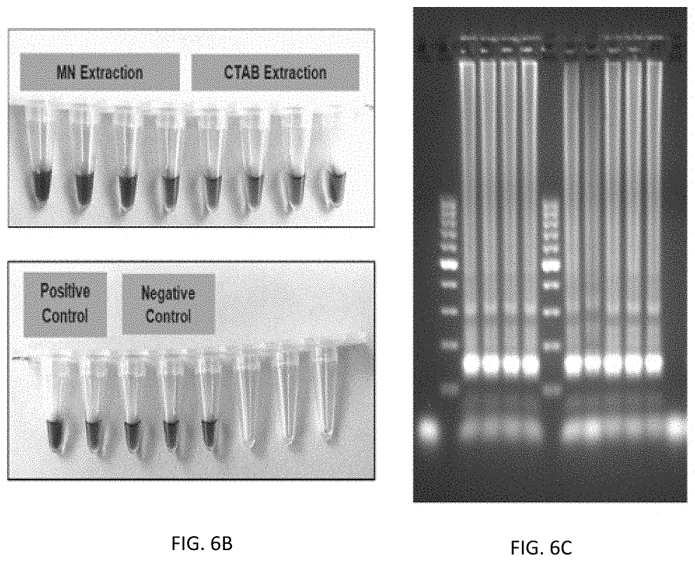

MN-extracted DNA is also directly applicable for LAMP amplification like PCR. To demonstrate that, DNA was extracted by MN patches from infected tomato leaves after 3 days of inoculation. Before DNA extraction, infected leaves were pre-disinfested by rinsing with a 5% bleach solution for a few seconds followed by rinsing with DI water twice. After extraction, real-time LAMP amplification reactions were performed using 1 μL of needle-extracted samples. See FIGS. 6A and 6B. All MN extracted samples were successfully amplified by the LAMP amplification and reaction tubes change color from violet to blue. See FIG. 6B. Finally, gel electrophoresis was performed to visualize the amplified DNA bands for all CTAB and MN-extracted samples. See FIG. 6C. These results confirm that MN-extracted DNA is LAMP amplifiable without additional purification.

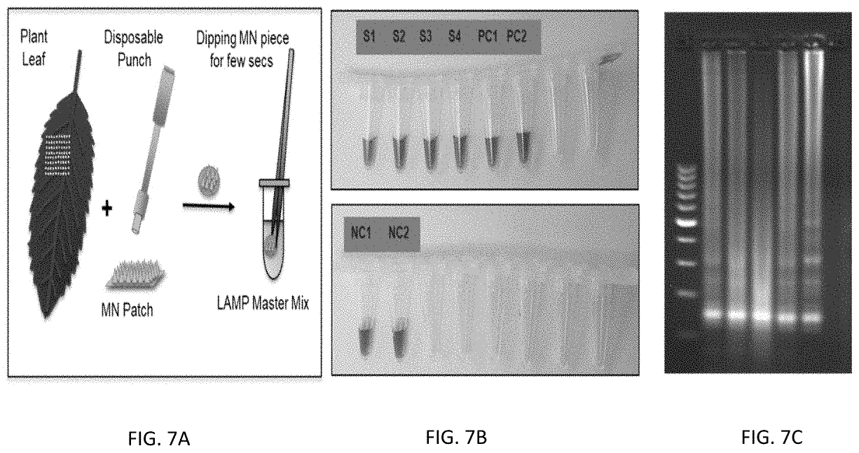

Instead of rinsing, MN-extracted DNA can also be transferred from needle tips to LAMP reaction mixture by dipping microneedles into the reaction solution for few seconds. To demonstrate that, MN patches were pressed gently onto infected leaf surfaces after surface disinfestation using 5% bleach solution. Then, MN patches were removed, and disposal laboratory punches were used to cut a small piece of patch into the circular shape. See FIG. 7A. Next, the small piece of MN patch was dipped into the LAMP master mixture for few seconds and removed. Amplification reactions were then started following normal heating conditions. All tested samples were successfully amplified by the LAMP amplification, where the HNB dye changed color from violet to sky blue. See FIG. 7B. Moreover, gel electrophoresis was performed to confirm the presence of desired amplicons. See FIG. 7C. These results suggested that direct dipping of MN pieces into LAMP reaction mixtures transferred sufficient amount of DNA templates for LAMP amplification.

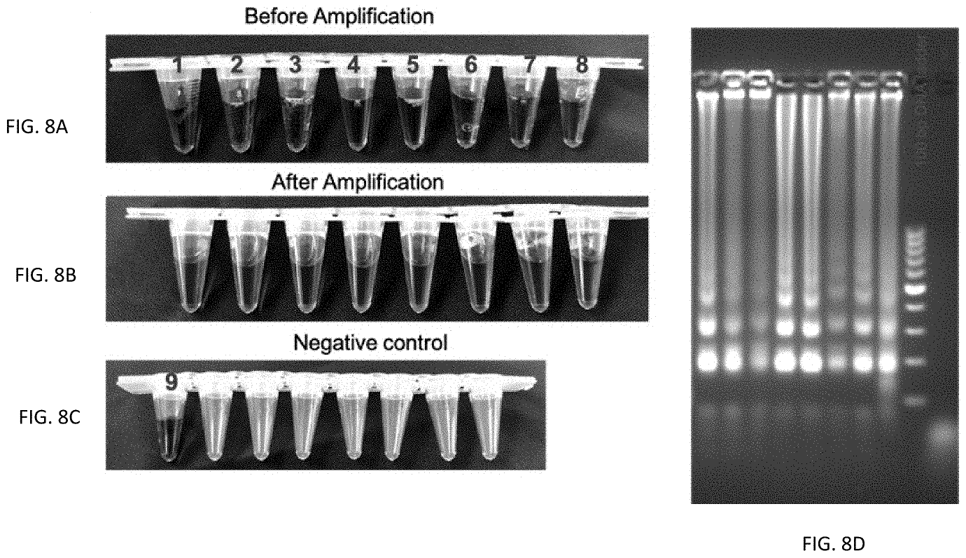

Moreover, it was also demonstrated that LAMP amplification reactions can be performed in the presence of MN patches, which indicates that the microneedle material, PVA, does not interfere with the LAMP assay process. See FIGS. 8A-8D. For that, MN patches were applied on infected leaves to extract P. infestans DNA. Then, MN patches were cut into small pieces and added into LAMP reaction tubes. See FIG. 8A. After the reaction, PVA patches were dissolved completely in the reaction mixtures, and the solution color was changed from violet to blue, indicating the successful amplification. See FIG. 8B. Gel electrophoresis further confirmed the successful amplification of P. infestans for all tested samples, even with dissolved PVA in the LAMP assay. See FIG. 8D.

Finally, a smartphone-based LAMP amplification platform has been developed for in-field extraction, amplification, and detection of P. infestans. See FIGS. 9A and 9B. In the device, a PDMS chip (˜10 mm×10 mm) was used in the sample cassette for running the LAMP assay. After DNA extraction, the MN patch can be dipped directly into the LAMP master mixture to deposit template DNA into the reaction chamber. See FIG. 9A. To heat LAMP reaction mixers in LAMP cassette, a battery powered, flexible polyamide heater has been included in the sample cartridge. See FIG. 9C. One the main advantages of this polyamide heater is that the surface temperature of the heater can be easily controller by changing input voltage (see FIG. 9D), eliminating the need to include a temperature controller in the device. During amplification, eight blue LEDs (wavelength=470 nm) were used in the reader device to excite the reaction chamber with a tilted illumination angle. The fluorescent signals were collected by an external lens placed in front of the smartphone camera, separated by a bandpass emission filter (543 nm±10 nm), and finally entered the smartphone camera for imaging. See FIG. 9B.

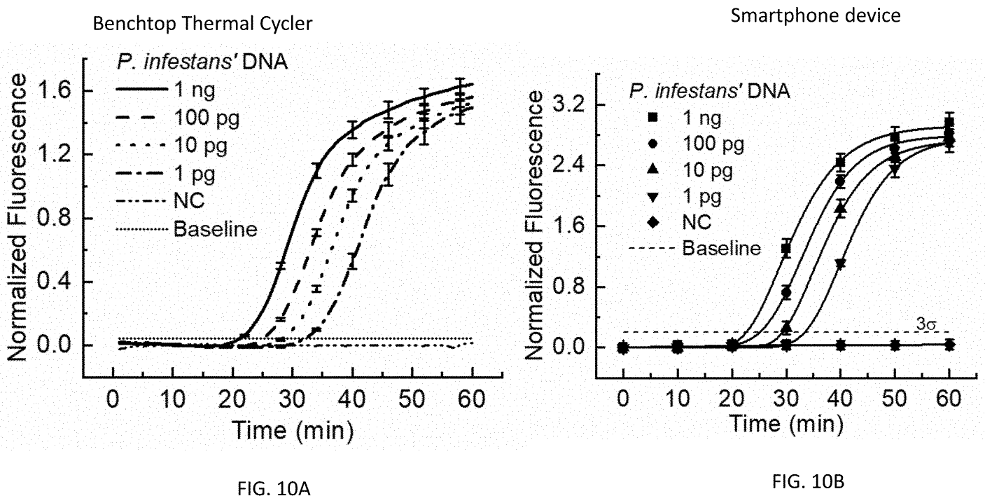

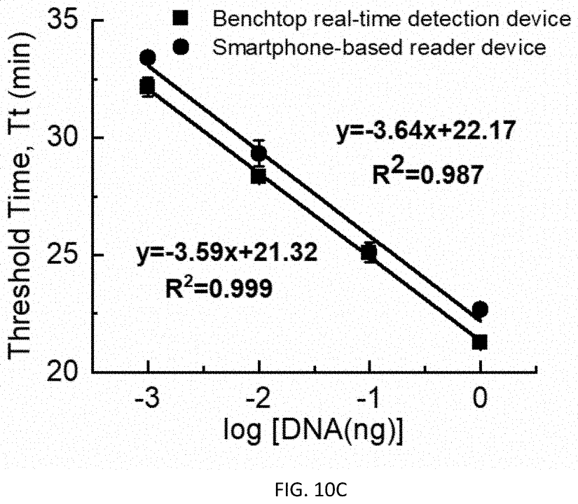

The performance of a smartphone-based LAMP amplification device was tested for P. infestans detection, and results were compared with those obtained from the benchtop PCR machine. See FIGS. 10A-10C. For running a real-time LAMP assay in a reader device, the purified P. infestans DNA was mixed with the LAMP reagents in the PDMS reaction chamber. The chamber was then sealed and inserted into the reader device and heated to 65° C. using a flexible polyamide heater. Images were captured using smartphone at a 10-min interval. See FIG. 10B. After the amplification reaction, the green channel intensities of the smartphone images were analyzed by ImageJ software. On the other hand, for running LAMP assay in benchtop PCR machine, samples were loaded into the conventional PCR tubes and heated to 65° C. in CFX Connect Real-Time PCR Detection System (Bio-Rad Laboratories, Hercules, Calif., United States of America, catalog no. 1855200). The fluorescence signals were captured at a 1-min interval. See FIG. 10A. For both devices (benchtop PCR machine vs. smartphone), a similar limit of detection (1 pg) was observed for P. infestans. See FIGS. 10A and 10B. Also, a linear relation between amplification threshold time (Tt) and logarithmic values of target concentrations was observed for both platforms. See FIG. 10C. Compared with the conventional benchtop thermal cycler, the smartphone-based amplification platform requires 2-3 more minutes on average to amplify the same amount of DNA/RNA, which, without being bound to any one theory, is believed to be due to the slow temperature ramping rate of the microheater. Potentially, this can be avoided by prewarming the LAMP cassette before loading the samples.

### Example 3

### RNA Extraction and Amplification by Conventional PCR

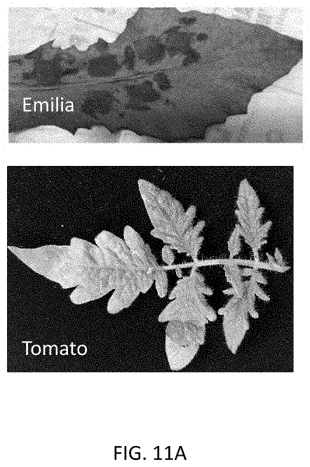

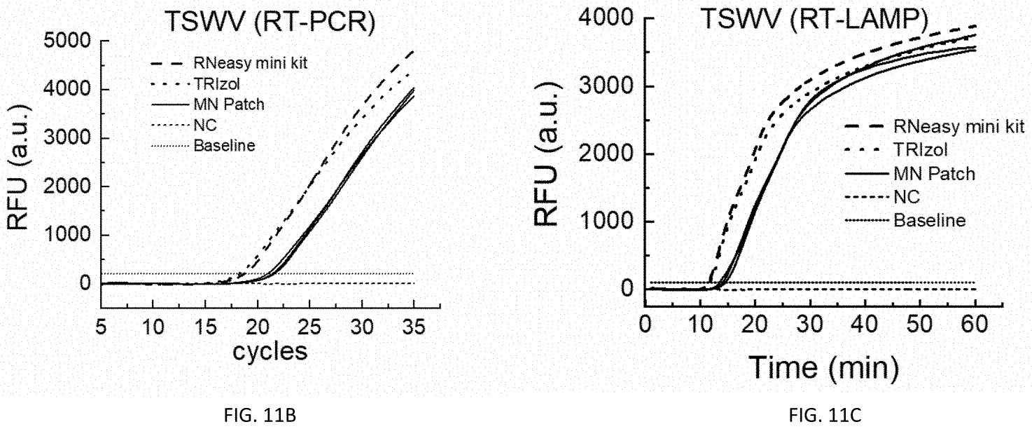

In addition to DNA extraction, the efficacy of MN patch for RNA extraction from plant tissues for the detection of viral infections. To do that, Emilia and tomato plant leaves were inoculated with tomato spotted wilt virus (TSWV), a devastating RNA virus that can be transmitted by thrips and infects thousands of different hosts, including agronomically important crops such as tomatoes and tobacco. See FIG. 11A. For RNA extraction and isolation, three different methods were tested side-by-side, namely a spin column-based RNA isolation kit (sold under the tradename RNEASY™ mini kit, Qiagen GMBH, Hilden, Germany), a conventional extraction protocol using a reagent sold under the tradename TRIZOL™ (Molecular Research Center, Inc., Cincinnati, Ohio, United States of America), and MN extraction according to the presently disclosed subject matter. For the first two methods, Emilia and tomato leaves were ground before extraction. For MN extraction, no leaf grinding is needed, and nuclease-free water was applied to rinse the MN patch after compression and collect RNA samples. All samples were later analyzed by the same reverse transcription-polymerase chain reaction (RT-PCR) and reverse transcription loop-mediated isothermal amplification (RT-LAMP) to validate the existence of viral RNA in the extracted samples. The results suggested that all three extraction methods have successfully extracted viral RNA. See FIGS. 11B and 11C. The MN extraction showed a slight delay in terms of amplification signals, which, without being bound to any one theory, is believed due to the complex sample matrix without any purification. If standard RNA purification steps (e.g., organic extraction, centrifugation, and participation, etc.) are also applied for MN-extracted samples, the threshold cycles (Ct) or threshold time (Tt) of the three methods are quite comparable to each other.

### Example 4

### Microneedle-Extracted Nucleic Acid for Sequencing and Genotyping

The presently disclosed microneedle patch can also be used as a low-lost and rapid sampling method for DNA sequencing and genotyping. Typically, 100 to 1000 nanograms (ng) of DNA are required for whole-genome or whole-exome sequencing. Targeted panels or amplicon-based sequencing can use as little as 1 to 10 ng of input material. Using a small patch in the size of a stamp and an array of 15×15 needles, the concentration of total DNA in the extracted solution (100 μL) is typically within the range of 20-50 ng/mL, as determined by NanoDrop measurement. The total DNA is then estimated to be around 500 to 1000 ng from a patch of 225 needle tips. It is also reasonable to assume the majority of the extracted DNA is host DNA from the plant. Therefore, the calculation indicates that it is feasible to perform whole-genome sequencing of the host or targeted sequencing to detect pathogens from the MN-extracted samples.

### Example 5

### Microneedle-Based Fungicide Delivery

Finally, the utility of MN patches for delivery of two different fungicides (a fungicide sold under the tradename PRESIDIO™ (Valent U.S.A., LLC; Walnut Creek, Calif., United States of America; also referred to herein as Fungicide 1) and a fungicide sold under the tradename RIDOMIL GOLD™ (Syngenta Participations AG Corporation; Basel Switzerland; also referred to herein as Fungicide 2) was tested.

### Fungicide Delivery—Fungicide 1:

The MN patches were used to deliver fungicide 1 (active ingredient (AI): fluopicolide, 39.5%) into tomato leaves for control of P. infestans. MN patches with a therapeutic dose (3 lb./A) of fungicide at 50 μl applied to the needles were pressed into detached tomato leaves. Control treatments included leaves treated with a microneedle patch with buffer applied to the needles (1:5 dilution of 0.5% Tween 80), leaves sprayed with buffer, or with a therapeutic dose of Fungicide 1 delivered as a spray. Leaves were allowed to rest for two days before being challenged with 0.5 mL of P. infestans sporangia solution at a concentration of 1000 sporangia/mL. Leaves were incubated under 12 h light for a week post-inoculation before sporangia counts were measured for each leaf.

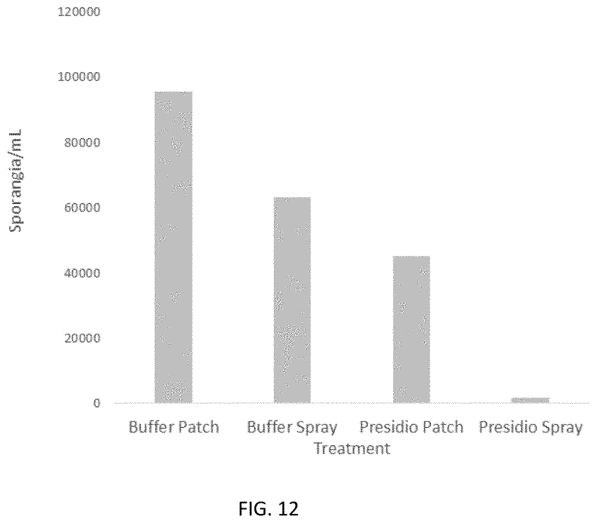

Leaves treated with buffer (patch or spray) showed higher sporangia counts compared to leaves treated with Fungicide 1. However, the sporangia count for leaves treated with a Fungicide 1 spray were much lower than leaves treated with Fungicide 1 on a patch (average 1667 vs 45000 sporangia). Leaves treated with Fungicide 1 on a patch showed only slightly less sporangia than leaves treated with buffer spray (63333 vs 45000 sporangia). See FIG. 12.

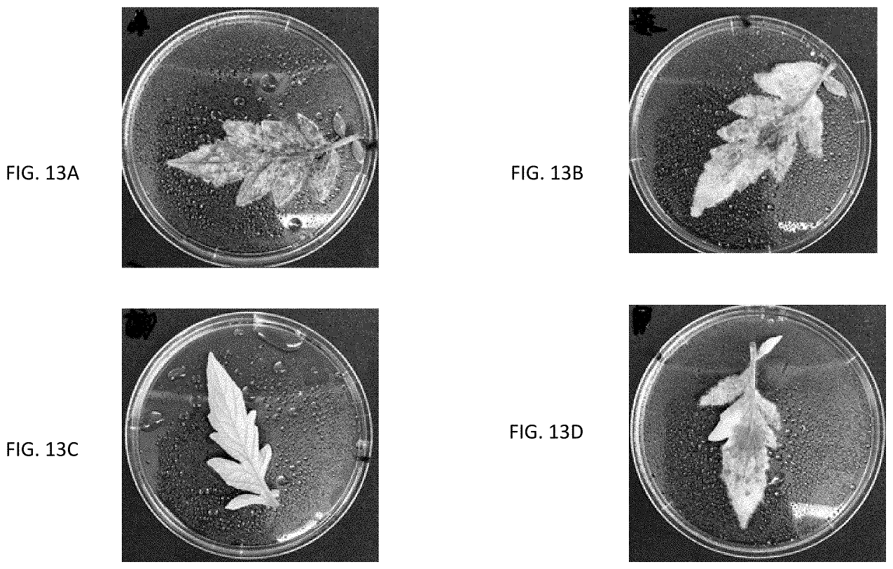

Photographs showed that sprayed fungicide resulted in a uniform treatment, whereas MN patch provided more localized treatment results. See FIGS. 13A-13D. It was observed on leaves treated with Fungicide 1 on a patch that there was a small area of nonsporulating/healthy tissue around the injection point, which likely resulted in the slightly lower sporangia counts. See FIG. 13D. When applying Fungicide 1 via the patch, it was observed that much of the liquid was pressed out along the surface of the leaf rather than being injected because of the pressure used to penetrate the leaf tissue. Without being bound to any one theory, it is likely that this healthier portion of the leaf received Fungicide 1 via the surface rather than direct injection by the microneedles.

### Fungicide Delivery: Fungicide 2:

Following evaluation of the use of microneedle patches with the use of Fungicide 1 and observing localized effectiveness, the use of the patches with a systemic fungicide was examined using the commercial formulation of Fungicide 2 (AI: mefenoxam 45.3%). With a standard application rate of 1 pt/A, the dosage amount for the area of a petri dish was calculated to be 7.25 μl. This volume was diluted into either 25 μl or 1 mL for application via microneedle patch or spray, respectively (dilution buffer: 1:5 dilution of 0.5% Tween 80). Microneedle patches with either a dose of 7.25 μl fungicide at 25 μl or 25 μl dilution buffer were applied to detached tomato leaves. In addition, leaves were sprayed with either 1 ml of fungicide (7.25 μl diluted into 1 mL) at the application rate or 1 mL buffer. Leaves were incubated under 12 hour/day light for 2 days before being challenged with 0.5 mL of P. infestans sporangia solution at a concentration of 1000 sporangia/mL. Leaves were incubated under 12 hour/day light for a week post-inoculation before sporangia counts were measured for each leaf. A total of 12 leaves were tested, three for each treatment.

An additional trial has been run with varying levels of Fungicide 2 dosage to assess the degree of control versus the amount of leaf damage observed. Solutions of Fungicide 2 were prepared at standard application rate (1 pt/A), half-standard application rate, and quarter standard application rate. An additional solution of buffer was also prepared as a control. Leaves were either sprayed with 1 ml of one of the four above solutions or treated with a microneedle patch using a dose of fungicide at the designated level within 25 μl. A total of 24 leaves were tested, 3 for each treatment (Fungicide 2 concentration/spray or patch).

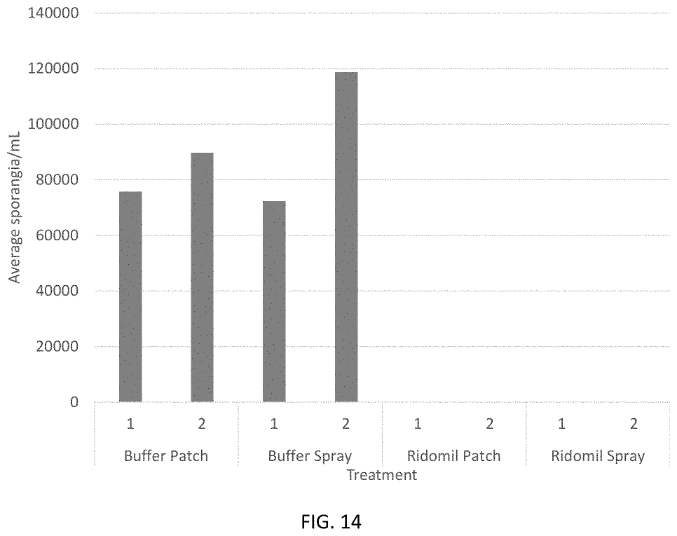

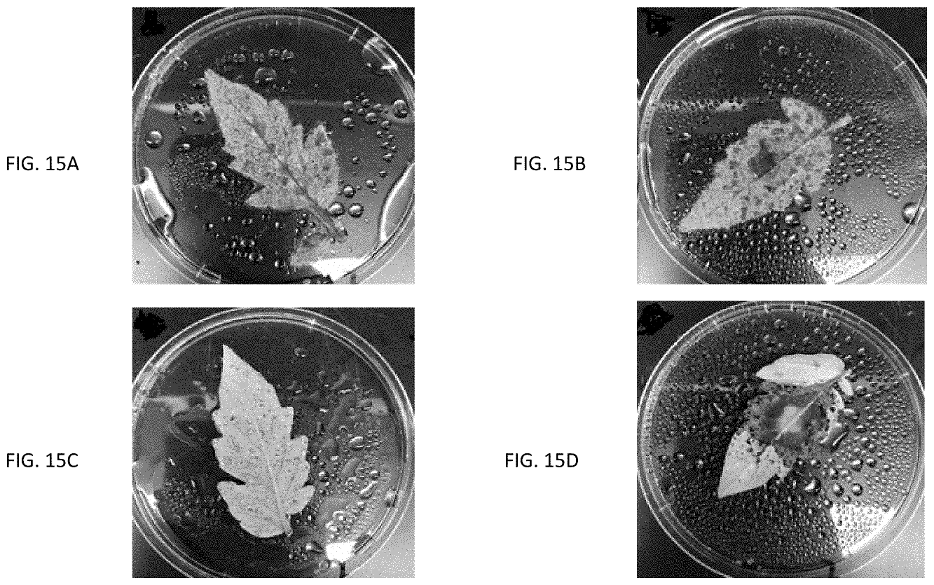

Two repetitions of the standard application rate experiment were completed. See FIGS. 14 and 15A-15D. In both experiments, application of Fungicide 2 using either a spray or patch method reduced the sporangia count to zero. See FIG. 14. However, it was observed that leaves treated with fungicide 2 applied via MN patch exhibited symptoms of phytotoxicity, including browning and chlorosis around the application site. See FIG. 15D. Some brown speckling was observed on leaves that received Fungicide 2 as a spray treatment, but not to the same degree as the leaves that received Fungicide 2 via patch. See FIG. 15C. To mitigate this side effect, reduced Fungicide 2 dosage was tested in the following experiments.

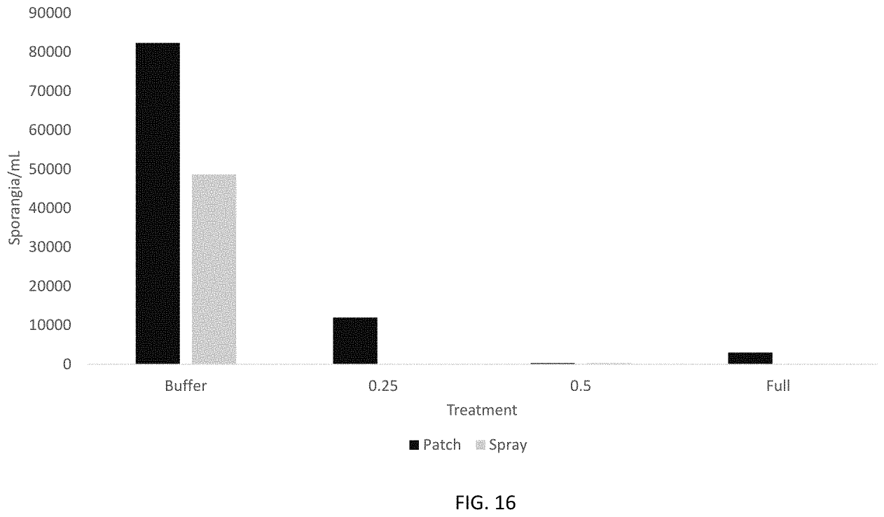

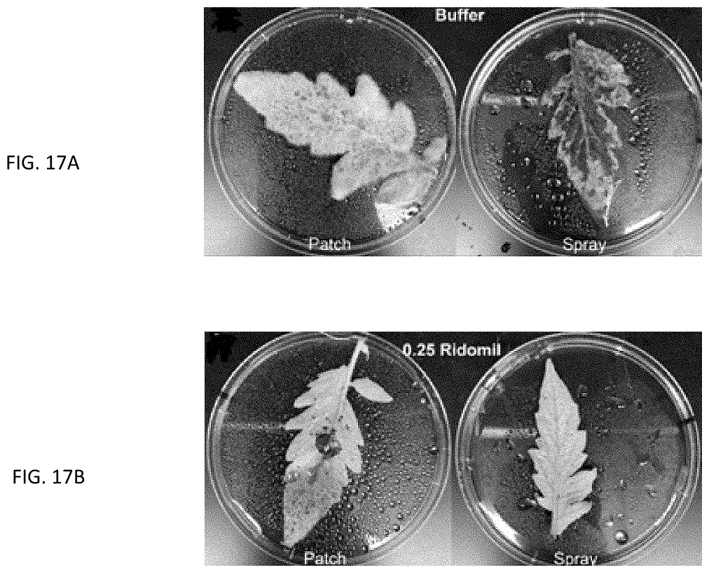

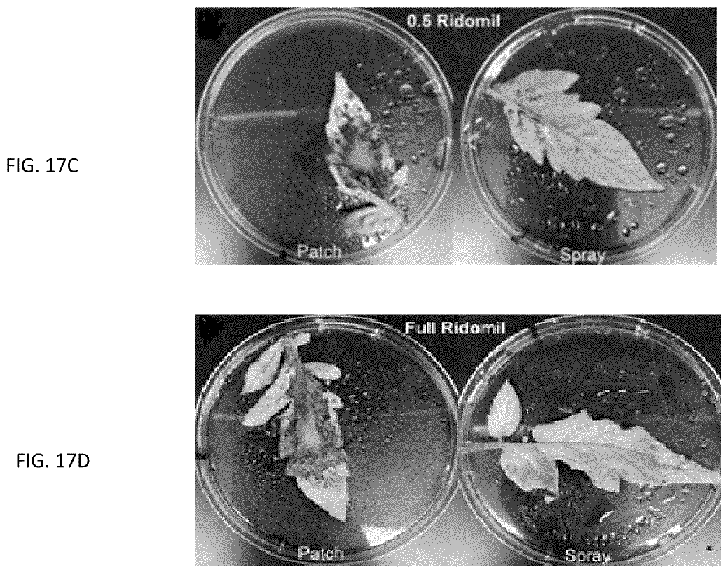

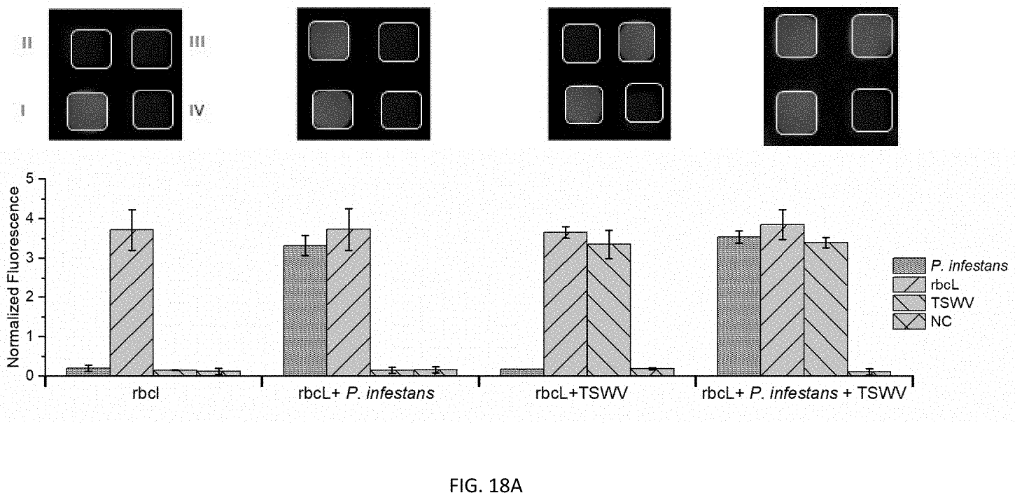

A repetition of the variable dosage experiment was performed. See FIGS. 16 and 17A-17D. Under the spray treatment, no sporangia were detected at the quarter and full rate of Fungicide 2 while a minimal number of sporangia were detected at the half rate level. See FIG. 16. Under the patch treatment, sporangia counts were significantly reduced compared to the buffer control, but some sporangia were detected especially for the quarter rate level. See FIG. 16. In one plate treated with a full Fungicide 2 dosage with a patch treatment, a secondary leaflet present on the leaf was noted to be generating sporangia, resulting in the higher level of sporangia measured. See FIG. 17D. Without being bound to any one theory, it is likely that the fungicide was unable to reach the secondary leaflet to provide protection, possibly due to damage to the leaf vascular system during harvesting. No sporangia were detected on the other two leaves treated with a full application of Fungicide 2 delivered via patch.

Accordingly, reduction of the amount of applied fungicide via MN patch appeared to reduce the amount of phytotoxic damage, but with some tradeoff via an increase in the amount of disease. Overall, results from these studies using Fungicide 2 indicate that MN patches can be used for delivery of systemic fungicides.

### Example 6

### Multiplexed Pathogen Detection with the MN-Smartphone Platform

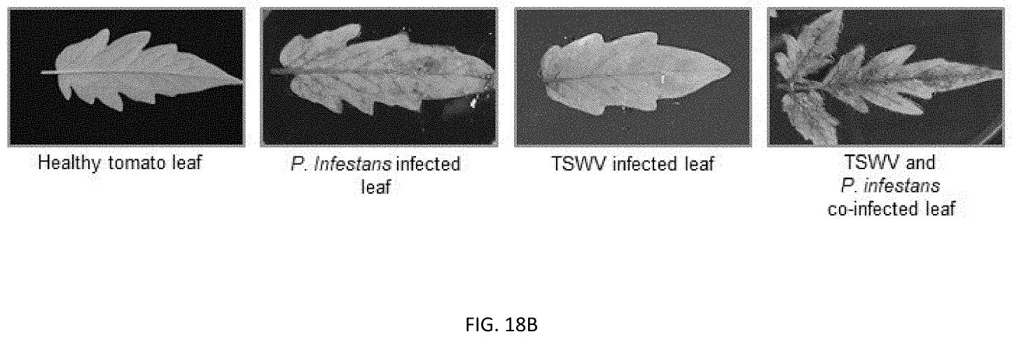

The presently disclosed microneedle patch and smartphone-based amplification platform can also be used to detect multiple plant pathogens simultaneously from a single infected leaf. See FIGS. 18A and 18B. In the LAMP cassette, three targets from a single MN extraction were amplified at the same time. See FIG. 18A. However, the number of targets can be easily increased by increasing the number of reaction chambers in the cassette. For multiplexed detection, nucleic acid targets were extracted from four different groups of tomato leaves: healthy controls (targeted for the rbcL gene), P. infestans infected leaves, TSWV-infected leaves, and co-infected leaves with both P. infestans and TSWV. FIG. 18B shows the photographs of a representative leaf sample from each group used in the experiment. For detection, different LAMP primers that were specific to the rbcL gene, P. infestans DNA, and TSWV RNA were added in three reaction chambers, and the remaining chamber was utilized as the negative control (NC: no LAMP primers). The representative fluorescence images captured after LAMP amplification for each group, clearly showing the successful detection of each targets in different sample groups. See FIG. 18A (top). Quantitatively, the green channel intensities of the captured smartphone images before and after amplification reactions were analyzed to compute the normalized fluorescence (=intensity change/initial intensity) for different reaction chamber, which confirms the positive singles in each image. See FIG. 18A (bottom).

### Example 7

### Summary of Examples 1-6

In summary, a multifunctional and miniaturized diagnostic platform comprising a microneedle patch and smartphone device is demonstrated for rapid nucleic acid extraction, amplification, detection, and delivery of therapeutic reagents. The MN patch effectively extracts both DNA and RNA from plant tissues, proteins, carbohydrates or other chemical constituents in a nondestructive fashion, which reduces sample preparation time from hours of a conventional extraction method to ˜1 min. The MN-extracted DNA is purification-free and directly applicable for amplification assays such as PCR and LAMP. It was demonstrated that MN extraction could be used to detect P. infestans DNA or TSWV RNA in infected plant leaves. A smartphone-based amplification and optical readout system can support isothermal nucleic acid amplification of extracted samples directly in the field. Finally, promising initial results of delivery of various fungicides by the MN patch to the plant tissues to suppress pathogen infections was demonstrated.

### REFERENCES

All references listed in the instant disclosure, including but not limited to those identified by reference numbers and that correspond to the reference numbers below, and also including but not limited to all patents, patent applications and publications thereof, scientific journal articles, and database entries are incorporated herein by reference in their entireties to the extent that they supplement, explain, provide a background for, or teach methodology, techniques, and/or embodiments employed herein.
1. Godfray, H. C. J. et al. Food security: the challenge of feeding 9 billion people. Science 327, 812-8 (2010).
2. World Development Report 2008: Agriculture for Development. (The World Bank, 2007). doi:10.1596/978-0-8213-6807-7
3. Cabanilla, L. S. Socio-economic and political concerns for GM foods and biotechnology adoption in the Philippines. AgBioForum 10, 178-183 (2007).
4. Fang, Y. & Ramasamy, R. P. Current and prospective methods for plant disease detection. Biosensors 5, 537-561 (2015).
5. Sankaran, S., Mishra, A., Ehsani, R. & Davis, C. A review of advanced techniques for detecting plant diseases. Comput. Electron. Agric. 72, 1-13 (2010).
6. López, M. M. et al. Innovative tools for detection of plant pathogenic viruses and bacteria. Int. Microbiol. 6, 233-243 (2003).
7. De Bary, A. Researches into the nature of the potato fungus, Phytophthora infestans. J. Bot. Paris 14, 105-126 (1876).
8. Berkele, M. J. Observations, botanical and physiological on the potato murrain. J. Hortic. Soc. London 1, 9-34 (1846).
9. Bourke, P. A. Emergence of potato blight, 1843-46. Nature 203, 805-808 (1964).
10. Hu, C.-H. et al. Recent Genotypes of Phytophthora infestans in the Eastern United States Reveal Clonal Populations and Reappearance of Mefenoxam Sensitivity. Plant Dis. 96, 1323-1330 (2012).
11. Fry, W. E. et al. The 2009 Late Blight Pandemic in the Eastern United States—Causes and Results. Plant Dis. 97, 296-306 (2013).
12. MOSKIN, J. Outbreak of fungus threatens tomato crop. Available at: https://www.nytimes.com/2009/07/18/nyregion/18tomatoes.html?_r=0. (Accessed: 31 Aug. 2018)
13. Haverkort, A. J. et al. Societal Costs of Late Blight in Potato and Prospects of Durable Resistance Through Cisgenic Modification. Potato Res. 51, 47-57 (2008).
14. Ristaino, J. B., Groves, C. T. & Parra, G. R. PCR amplification of the Irish potato famine pathogen from historic specimens. Nature 411, 695-697 (2001).
15. Haas, B. J. et al. Genome sequence and analysis of the Irish potato famine pathogen Phytophthora infestans. Nature 461, 393-398 (2009).
16. Trout, C. L., Ristaino, J. B., Madritch, M. & Wangsomboondee, T. Rapid Detection of Phytophthora infestans in Late Blight-Infected Potato and Tomato Using PCR. Plant Dis. 81, 1042-1048 (1997).
17. Koo, C. et al. Development of a real-time microchip PCR system for portable plant disease diagnosis. PLoS One 8, e82704 (2013).
18. Julich, S. et al. Development of a lab-on-a-chip device for diagnosis of plant pathogens. Biosens. Bioelectron. 26, 4070-4075 (2011).
19. Murray, M. G. & Thompson, W. F. Rapid isolation of high molecular weight plant DNA. Nucleic Acids Res. 8, 4321-5 (1980).
20. Wang, H., Qi, M. & Cutler, A. J. A simple method of preparing plant samples for PCR. Nucleic Acids Res. 21, 4153-4154 (1993).
21. Agdia—Sample Extraction Bag, SEB. Available at:

https://orders.agdia.com/sample-bag-extract-acc-00936. (Accessed: 25 Jul. 2018)
22. EuroBlight. Protocol for sampling Phytophthora infestans DNA using FTA cards 2018. Available at: http://euroblightnet/fileadmin/euroblight/Monitoring_2013_project/EuroBlig ht_sampling_2018. pdf. (Accessed: 29 Jun. 2018)
23. Ndunguru, J. et al. Application of FTA technology for sampling, recovery and molecular characterization of viral pathogens and virus-derived transgenes from plant tissues. Virol. J. 2, 45 (2005).
24. Brown, R. B. & Audet, J. Current techniques for single-cell lysis. J. R. Soc. Interface 5, S131-S138 (2008).
25. Kim, J., Johnson, M., Hill, P. & Gale, B. K. Microfluidic sample preparation: cell lysis and nucleic acid purification. Integr. Biol. 1, 574 (2009).
26. Nan, L., Jiang, Z. & Wei, X. Emerging microfluidic devices for cell lysis: a review. Lab chip 14, 1060 (2014).
27. Wu, J., Kodzius, R., Cao, W. & Wen, W. Extraction, amplification and detection of DNA in microfluidic chip-based assays. Microchim. Acta 181, 1611-1631 (2014).
28. Ristaino, J. B., Saville, A., Paul, R., Cooper, D. & Wei, Q. Detection of Phytophthora infestans by LAMP, real-time LAMP and droplet digital PCR. Plant Dis. PDIS-06-19-1186-RE (2019). doi:10.1094/PDIS-06-19-1186-RE
29. Goto, M., Honda, E., Ogura, A., Nomoto, A. & Hanaki, K.-I. Colorimetric detection of loop-mediated isothermal amplification reaction by using hydroxy naphthol blue. Biotechniques 46, 167-172 (2009).
30. Ye, Y. et al. Synergistic Transcutaneous Immunotherapy Enhances Antitumor Immune Responses through Delivery of Checkpoint Inhibitors. ACS Nano 10, 8956-8963 (2016).
31. Larrañeta, E., Lutton, R. E. M., Woolfson, A. D. & Donnelly, R. F. Microneedle arrays as transdermal and intradermal drug delivery systems: Materials science, manufacture and commercial development. Mater. Sci. Eng. R Reports 104, 1-32 (2016).
32. Demir, Y. K., Akan, Z. & Kerimoglu, O. Characterization of Polymeric Microneedle Arrays for Transdermal Drug Delivery. PLoS One 8, e77289 (2013).
33. Sullivan, S. P. et al. Dissolving polymer microneedle patches for influenza vaccination. Nat. Med. 16, 915-920 (2010).
34. Yu, J. et al. Microneedle-array patches loaded with hypoxia-sensitive vesicles provide fast glucose-responsive insulin delivery. Proc. Natl. Acad. Sci. U.S.A. 112, 8260-5 (2015).
35. Wang, C., Ye, Y., Hochu, G. M., Sadeghifar, H. & Gu, Z. Enhanced Cancer Immunotherapy by Microneedle Patch-Assisted Delivery of Anti-PD1 Antibody. Nano Lett. 16, 2334-2340 (2016).
36. Wang, J. et al. Core-Shell Microneedle Gel for Self-Regulated Insulin Delivery. ACS Nano 12, 2466-2473 (2018).
37. Varma, A., Padh, H. & Shrivastava, N. Plant genomic DNA isolation: An art or a science. Biotechnol. J. 2, 386-392 (2007).
38. Edwards, K., Johnstone, C. & Thompson, C. A simple and rapid method for the preparation of plant genomic DNA for PCR analysis. Nucleic Acids Res. 19, 1349 (1991).
39. Fazekas, A. J., Burgess, K. S., Kesanakurti, P. R., Graham, S. W. & Newmaster, S. G. Multiple Multilocus DNA Barcodes from the Plastid Genome Discriminate Plant Species Equally Well. PLoS One 3, 2802 (2008).
40. Ristaino, J. B., Ivors, K., Bonants, P., Gómez-Alpizar, L. & Blanco-Meneses, M. Rapid Diagnostic Tools for Phytophthora on Horticultural Crops. (Horticultural Crops. USAID Hort CRSP Workshop, San Jose Costa Rica, Jun. 28-Jul. 5, 2010).

While the methods, compositions, and systems have been described herein in reference to specific aspects, features, and illustrative embodiments, it will be appreciated that the utility of the subject matter is not thus limited, but rather extends to and encompasses numerous other variations, modifications and alternative embodiments, as will suggest themselves to those of ordinary skill in the field of the present subject matter, based on the disclosure herein. Various combinations and sub-combinations of the structures and features described herein are contemplated and will be apparent to a skilled person having knowledge of this disclosure. Any of the various features and elements as disclosed herein can be combined with one or more other disclosed features and elements unless indicated to the contrary herein. Correspondingly, the subject matter as hereinafter claimed is intended to be broadly construed and interpreted, as including all such variations, modifications and alternative embodiments, within its scope and including equivalents of the claims.

What is claimed is:

1. A method of extracting an analyte from a biological sample comprising a plant tissue or an animal tissue, wherein when the biological sample comprises a plant tissue, the plant tissue is a soft plant tissue, optionally wherein the soft plant tissue is selected from the group consisting of an immature plant tissue, a flower, a seedling, a plant leaf, a tuber, a fruit and a stem; the method comprising:
contacting the plant tissue or the animal tissue with a microneedle, wherein said microneedle comprises a body having a base and a tip;
removing the microneedle from contact with the plant tissue or the animal tissue, wherein the removed microneedle comprises absorbed analyte extracted from the plant tissue or the animal tissue; and
optionally collecting the analyte.

2. The method of claim 1, wherein the microneedle has a fracture force of about 1 newton (N) or less, optionally about 0.1 N to about 1 N.

3. The method of claim 1, wherein the microneedle body has a length of about 50 microns (μm) to about 1500 μm; and wherein the tip has a diameter of about 1 μm to about 10 μm; optionally wherein the microneedle has a length of about 800 μm and a tip diameter of about 5 μm.

4. The method of claim 3, wherein the microneedle body has a tapered, conical, and/or pyramidal shape, wherein said microneedle body is hollow or solid, and wherein the base of the microneedle has a diameter that is greater than the diameter of the tip of the microneedle, optionally wherein the base has a diameter that is about 10 μm to about 500 μm, further optionally wherein the base has a diameter that is about 150 μm.

5. The method of claim 1, wherein the body of the microneedle comprises, consists essentially of, or consists of a polymer, optionally wherein the polymer is a swellable and/or hydrophilic polymer.

6. The method of claim 5, wherein the body of the microneedle comprises, consists essentially of, or consists of a hydrophilic, swellable polymer selected from the group consisting of polyvinyl alcohol (PVA), crosslinked hyaluronic acid (HA), crosslinked polyacrylic acid (PAA), chitosan, and a copolymer thereof.

7. The method of claim 5, wherein the polymer is a charge-switchable polymer that is positively charged when exposed to a first pH range and uncharged when exposed to a second pH range; optionally wherein the charge-switchable polymer is positively charged when exposed to a pH below about 7; further optionally wherein said polymer is chitosan.

8. The method of claim 1, wherein the method is free of the use of suction.

9. The method of claim 1, wherein the analyte comprises a DNA, a RNA, a protein, carbohydrate or other chemical constituent and/or a small molecule, wherein said DNA, RNA, and/or protein is native to the plant tissue or animal tissue and/or to a pathogen or pest.

10. The method of claim 1, wherein the contacting comprises:
placing the tip of the microneedle on an outer surface of the plant tissue or the animal tissue, optionally wherein the outer surface is a cuticle layer or an epidermal layer of a plant tissue or a skin surface of an animal; and
exerting a force on the microneedle sufficient to puncture the outer surface of the plant tissue or the animal tissue with the tip of the microneedle, thereby bringing at least a portion of the body of the microneedle into contact with inner cells of the plant tissue or the animal tissue.

11. The method of claim 1, wherein collecting the analyte comprises: contacting the microneedle with a liquid in which the analyte is soluble, thereby dissolving the absorbed analyte in the liquid and removing it from the microneedle; and
collecting the liquid comprising the dissolved analyte.

12. The method of claim 11, wherein the analyte comprises one or more nucleic acid and the liquid is a nucleic acid extraction buffer solution, optionally Tris-EDTA or nuclease-free water.

13. The method of claim 1, wherein contacting with a microneedle comprises contacting the outer surface of the plant tissue or the animal tissue with a plurality of microneedles, optionally wherein the plurality of microneedles is provided in an array format.

14. The method of claim 1, wherein the method is performed in about 1 minute or less.

15. The method of claim 1, wherein the method further comprises analyzing the analyte, optionally wherein the analyzing comprises identifying, sequencing, and/or quantifying the analyte.

16. The method of claim 1, wherein the biological sample comprises a plant tissue, optionally a plant leaf.

17. The method of claim 16, wherein said plant tissue is from a plant cultivated as part of an agricultural crop for use as food for humans or other animals, as fiber, as energy, or for the production of an industrial or consumer good.

18. A method of detecting a pathogen or pest in a plant or an animal, wherein the method comprises:
providing a microneedle or microneedle patch comprising one or more microneedles, wherein the microneedle or each microneedle of the microneedle patch has a body with a base and a tip, optionally wherein each microneedle body comprises, consists essentially of, or consists of a polymer, optionally a swellable and/or hydrophilic polymer;
contacting the microneedle or microneedle patch with a plant or animal tissue, wherein said plant tissue is a soft plant tissue, optionally wherein the soft plant tissue is a plant leaf;
removing the microneedle or microneedle patch from contact with the plant or animal tissue, wherein the removed microneedle or one or more microneedles of the removed microneedle patch comprise a foreign analyte extracted from the plant or animal tissue, wherein said foreign analyte is associated with the pathogen or pest, optionally wherein the foreign analyte is a nucleic acid and/or protein from the pathogen or pest;
optionally collecting the foreign analyte from the microneedle or microneedle patch; and
analyzing the foreign analyte to determine the presence of the pathogen or pest.

19. The method of claim 18, wherein the method comprises detecting a pathogen selected from the group consisting of a viral pathogen, a fungal pathogen, a bacterial pathogen, and an oomycete pathogen.

20. The method of claim 19, where the pathogen is a fungal pathogen, a bacterial pathogen, or an oomycete pathogen; and the foreign analyte is DNA or RNA.

21. The method of claim 19, where the pathogen is a viral pathogen and the foreign analyte is viral RNA.

22. The method of claim 18, wherein the foreign analyte is a nucleic acid and the analyzing comprises nucleic acid amplification, optionally wherein the amplification comprises a technique selected from the group consisting of polymerase chain reaction (PCR), reverse transcriptase PCR (RT-PCR), ligase chain reaction (LCR), transcription-based amplification, self-sustained sequence replication (3SR), loop-mediated isothermal amplification (LAMP), reverse transcriptase LAMP (RT-LAMP), nucleic acid sequence-base amplification (NASBA), rolling circle amplification (RCA), ligation-enabled padlock RCA, and CRISPR cassette; further optionally wherein the nucleic acid amplification is an isothermal amplification technique.

23. The method of claim 18, wherein the foreign analyte comprises a nucleic acid and the analyzing comprises sequencing and/or genotyping, optionally wherein the genotyping comprises detection of one or more marker of interest selected from an allele; a locus, optionally a quantitative trait locus (QTL); a microsatellite, optionally a short tandem repeat (STR) or a simple sequence repeat (SSR); a single nucleotide polymorphism (SNP); a single feature polymorphism (SFP); an insertion/deletion polymorphism; a restriction-fragment-length polymorphism (RFLP); an amplified fragment length polymorphism (AFLP); and a CRISPR cassette.

24. The method of claim 18, where the contacting comprises:
placing the tip of the microneedle or a tip of one or more microneedles of the microneedle patch on an outer surface of the plant tissue or animal tissue, optionally wherein the outer surface is a cuticle or epidermal layer of the plant tissue, optionally a plant leaf, or a skin surface of an animal, optionally a human; and
exerting a force on the microneedle or microneedle patch sufficient to puncture said outer surface, thereby bringing at least a portion of the body of the microneedle or of one or more microneedles of the microneedle patch into contact with inner cells of the plant tissue or the animal tissue.

25. The method of claim 18, wherein collecting the foreign analyte comprises:
contacting the microneedle or microneedle patch with a liquid in which the foreign analyte is soluble, thereby dissolving the absorbed foreign analyte in the liquid and removing it from the microneedle or microneedle patch; and
collecting the liquid comprising the dissolved foreign analyte.

26. The method of claim 18, wherein the analyzing is free of cell lysis and/or nucleic acid purification.

27. The method of claim 18, wherein the microneedle patch comprises between 1 and 1,000 microneedles, inclusive.

28. The method of claim 18, wherein the method comprises detecting any plant pathogen in a plant, optionally wherein the plant pathogen is selected from Phytophthora infestans, Xanthomonas perforans, Altermaria linariae, and tomato spotted wilt virus (TSWV).

29. A method of genotyping a plant or animal, the method comprising:
providing a microneedle or microneedle patch comprising one or more microneedles, wherein the microneedle or each microneedle of the microneedle patch has a body with a base and a tip, optionally wherein each microneedle body comprises, consists essentially of, or consists of a polymer, optionally a swellable and/or hydrophilic polymer;
contacting the microneedle or microneedle patch with a plant or animal tissue, wherein the plant tissue is a soft tissue, optionally where the soft tissue is a plant leaf;
removing the microneedle or microneedle patch from contact with the plant or animal tissue, wherein the removed microneedle or one or more microneedle of the removed microneedle patch comprise a native analyte, optionally a native nucleic acid and/or protein, extracted from the plant or animal tissue;
optionally collecting the native analyte from the microneedle or microneedle patch; and
analyzing the native analyte, thereby genotyping the plant or animal.

30. The method of claim 29, wherein a native nucleic acid is extracted from the plant or animal tissue, and the analyzing comprises nucleic acid amplification, optionally wherein the amplification comprises a technique selected from the group consisting of polymerase chain reaction (PCR), reverse transcriptase PCR (RT-PCR), ligase chain reaction (LCR), transcription-based amplification, self-sustained sequence replication (3SR), loop-mediated isothermal amplification (LAMP), reverse transcriptase LAMP (RT-LAMP), rolling circle amplification (RCA), ligation-enabled padlock RCA, nucleic acid sequence-base amplification (NASBA), and CRISPR cassette.

31. The method of claim 29, wherein the analyzing comprises sequencing and/or genotyping, optionally wherein the analyzing comprises detection of one or more marker of interest selected from an allele; a locus, optionally a quantitative trait locus (QTL); a microsatellite, optionally a short tandem repeat (STR) or a simple sequence repeat (SSR); a single nucleotide polymorphism (SNP); a single feature polymorphism (SFP); an insertion/deletion polymorphism; a restriction-fragment-length polymorphism (RFLP); an amplified fragment length polymorphism (AFLP); and a CRISPR cassette.

32. The method of claim 29, wherein the contacting comprises:
placing a tip of the microneedle or of one or more microneedle of the microneedle patch on an outer surface of the plant or animal tissue, optionally wherein the outer surface is a cuticle or epidermal layer of a plant tissue, optionally a plant leaf, or a skin surface of an animal, optionally a human; and
exerting a force on the microneedle or microneedle patch sufficient to puncture said outer surface, thereby bringing at least a portion of the body of the microneedle or of one or more microneedle of the microneedle patch into contact with inner cells of the plant or animal tissue.

33. The method of claim 29, wherein collecting the native analyte comprises:
contacting the microneedle or microneedle patch with a liquid in which the native analyte is soluble, thereby dissolving the native analyte in the liquid and removing it from the microneedle or microneedle patch; and
collecting the liquid comprising the dissolved native analyte.

34. The method of claim 29, wherein the analyzing is free of cell lysis and/or nucleic acid purification.

35. The method of claim 29, wherein the microneedle patch comprises between 1 and 1,000 microneedles, inclusive.

36. A method of delivering a substance of interest to a plant; the method comprising:
providing a microneedle or microneedle patch comprising one or more microneedles, optionally a plurality of microneedles, wherein the microneedle or each microneedle of the microneedle patch comprises a body comprising a base and a tip, and wherein the body of the microneedle or the body of at least one body of the microneedle patch comprises or is coated with a substance of interest; and
contacting the microneedle or microneedle patch with a plant tissue, wherein said plant tissue is a soft tissue, optionally a plant leaf, thereby delivering the substance of interest to the plant.

37. The method of claim 36, wherein the substance of interest comprises one or more of the group selected from a pesticide, DNA, RNA, a protein, a peptide, an antigen for stimulating the plant immune system, a vector, a plasmid, a CRISPR cassette, a biological cell, a biological cell component, and/or a vesicle.

38. The method of claim 36, wherein the substance of interest comprises a pesticide, optionally wherein the pesticide is selected from a systemic fungicide, a translaminar fungicide, an herbicide, an insecticide, a biological control agent, and combinations thereof.

39. The method of claim 36, wherein the body of each microneedle comprises, consists essentially of, or consists of a polymer.

40. The method of claim 39, wherein the porosity of the polymer is tailored to provide a desired rate of delivery of the substance of interest or wherein said polymer is biodegradable.

41. A system for detecting an analyte of interest in a biological sample, optionally wherein the analyte of interest comprises a native analyte or a foreign analyte associated with a pathogen or pest, or any combination thereof, the system comprising:
a microneedle or microneedle patch configured to obtain an analyte extract from the sample, wherein said microneedle patch comprises one or more microneedles, optionally a plurality of microneedles, configured in an array, wherein the microneedle or each microneedle of the microneedle patch comprises a body comprising a base and a tip, optionally wherein each microneedle body comprises, consists essentially of, or consists of a swellable and/or hydrophilic polymer;
a receiver configured to receive a reaction mixture comprising (i) one or more reagents for detection of an analyte from the sample and (ii) the microneedle, the microneedle patch or an extract from the biological sample obtained using the microneedle or microneedle patch; and
a detector for detecting a signal from the reaction mixture associated with a reaction or interaction between an analyte and at least one of the one or more reagents;
wherein the biological sample is an animal and the microneedle or microneedle patch is configured to obtain an analyte extract from the animal by insertion and removal from a skin surface of the animal; or
wherein the biological sample is a soft tissue from a plant and the microneedle or microneedle patch is configured to obtain an analyte extract from the plant by insertion and removal from the soft tissue of the plant, optionally wherein the soft tissue is a plant leaf.

42. The system of claim 41, wherein the analyte extract comprises a nucleic acid, and wherein the one or more reagents comprise a polymerase and one or more nucleic acid primers for amplification of a nucleic acid associated with the one or more nucleic acids of interest.

43. The system of claim 42, wherein the analyte extract comprises a RNA, and wherein the one or more reagents further comprise a reverse transcriptase.

44. The system of claim 42, wherein the one or more reagents further comprise one or more fluorescent or colorimetric dyes, optionally wherein the one or more fluorescent or colorimetric dyes comprises hydroxynaphthol blue (HNB).

45. The system of claim 41, wherein the system further comprises an attachment configured to position the receiver with respect to the detector.

46. The system of claim 41, wherein the detector comprises a camera configured to capture an image of the receiver or a portion thereof.

47. The system of claim 46, wherein the detector comprises a consumer electronics device having a camera configured to capture an image of the receiver and one or more light sources configured to illuminate the receiver, optionally wherein the consumer electronics device is a smart phone or a tablet.

48. The system of claim 47, wherein the one or more light sources comprise one or more light emitting diodes (LEDs) or a laser diode.

49. The system of claim 41, wherein the receiver comprises a polydimethylsiloxane (PDMS) chamber or chip.

50. The system of claim 41, wherein the system further comprises a heating device configured to heat the receiver to a pre-determined temperature for a pre-determined period of time, optionally wherein the heating device is a flexible, polyamide heating device or a self-heating device or pad.

51. The system of claim 41, wherein the system further comprises one or more battery, optionally wherein the one or more battery is configured to provide power to a heating device configured to heat the receiver and/or one or more light source associated with the detector.

52. A method of detecting a presence and/or an amount of one or more analyte of interest in a biological sample, wherein the method comprises use of the system of claim 41.

53. The method of claim 54, wherein the one or more analytes of interest are associated with a pathogen or pest.
